(19) United States
(12) Patent Application Publication
(10) Pub. No.: US 2025/0180544 A1
(43) Pub. Date: Jun. 5, 2025

(54) ARTIFICIAL SERTOLI CELLS AND METHOD FOR THEIR PRODUCTION

(71) Applicant: The Regents of the University of Michigan, Ann Arbor, MI (US)

(72) Inventors: Saher Sue Hammoud, Ann Arbor, MI (US); Veda Yadagiri, Ann Arbor, MI (US); Umesh Kumar, Ann Arbor, MI (US)

(21) Appl. No.: 18/840,007

(22) PCT Filed: Feb. 22, 2023
(86) PCT No.: PCT/US2023/013608

§ 371 Date: Aug. 20, 2024

Related U.S. Application Data
(60) Provisional application No. 63/312,528, filed on Feb. 22, 2022.

(51) Int. Cl. G01N 33/50; C12N 5/071; C12N 5/076

(52) U.S. Cl. CPC G01N 33/5091; C12N 5/061; C12N 5/0683; G01N 33/5023; G01N 33/5082; C12N 2501/105; C12N 2501/11; C12N 2501/119; C12N 2501/155; C12N 2501/31; C12N 2501/998; C12N 2506/02; C12N 2533/92

(57) ABSTRACT

The present invention relates to in vitro methods for production of Sertoli cells and related organoids. The Sertoli cells and testis like organoids may be used for therapeutic purposes including facilitation of the production of spermatogonia from pro-spermatogonia stem cells.

### CROSS-REFERENCE TO RELATED APPLICATION

The present application claims priority to U.S. Provisional Patent Application Ser. No. 63/312,528, filed Feb. 22, 2022 the contents of which are hereby incorporated by reference in its entirety.

### STATEMENT REGARDING FEDERALLY SPONSORED RESEARCH OR DEVELOPMENT

This invention was made with government support under HD091949 awarded by the National Institutes of Health. The government has certain rights in the invention.

### FIELD OF THE INVENTION

The present invention relates to in vitro methods for production of Sertoli cells and related organoids.

### BACKGROUND OF THE INVENTION

Infertility is a rapidly rising crisis worldwide. One percent of all reproductive age couples (ages 20-50) globally suffer from infertility, and in roughly 50% of cases the cause is male factor infertility. In the US, 300,000 men have nonobstructive azoospermia (i.e., lacking any germ or mature sperm cells) and most are of unknown genetic origin. Traditional treatment options, such as in vitro fertilization (IVF) or intracytoplasmic sperm injection (ICSI), require sperm, and leave these men with no therapies. In addition to adult infertility, each year in the US ˜10,000 prepubertal boys develop cancers and require gonadotoxic treatments, such as chemotherapy and radiation (1). Furthermore, ˜1,000 pediatric patients with blood and immune deficiencies and autoimmune disorders receive myeloablative conditioning prior to bone marrow transplantation, which is also gonadotoxic (2). These treatments—with alkylating chemotherapeutic agents, total body irradiation (3), and gonadal radiation (4)—put patients at a significant risk of infertility due to damage to both somatic and germ cell populations in the testis. However, with the advancements in medicine 85% of these children will be cured and, upon reaching adulthood they desire to have their own children (5). Since gametes are needed for any reproductive therapy, developing methods to reconstitute germ cell development in vitro or in vivo for these patients remains a clinical challenge, and will continue to be the focus of the reproductive biology community for the coming decades. Successful generation of patient-derived, testis-like somatic cells in vitro will provide a novel cell-based therapy for genetic and iatrogenic (i.e., induced by treatment) forms of male infertility. Such a treatment is also applicable to restore endocrine function in aging men, those with hypogonadism, or patients with gender dysphoria. Further, if in vitro derived cells or cell communities (sometimes called organoids) can recapitulate the natural spermatogenesis processes, they can be used as a new experimental system to screen for new classes of male contraceptives.

The current race for cell-based treatment has centered on the development of germ cell-like precursors known as primordial germ cell-like cells (PGCLCs). While PGCLCs have been derived in vitro from mouse and human embryonic stem cells (ESCs), they cannot be maintained in vitro (6). Only when transplanted into mouse neonatal testis can these mouse PGCLCs develop into sperm, which can then be used to make live born pups. Furthermore, when female and male mouse PGCLCs were reconstituted with fetal gonadal tissue in vitro, successful reconstitution of the oogenesis and spermatogenesis program was possible, but the frequency of live born pups obtained from the in vitro derived germ cells is roughly ˜ 1-3% (6-9). These preliminary findings are exciting and underscore the importance of somatic cells for the execution of the gametogenesis program. In similar experiments using human or rhesus PGCLCs with mouse embryonic gonadal tissue from three research groups it was shown that when the female and male human hPGCLCs or male rhesus PGCLCs were combined with mouse fetal ovarian tissue or testis tissue, respectively, the PGCLC initiated differentiation, but failed to initiate meiosis (9-11). These observations indicate that somatic and germ cell sources must be compatible (i.e., from the same species) to initiate meiosis.

Utilizing somatic cells from human fetal testis to aid the progression of in vitro derived human PGCLCs has ethical and technical limitations related to fetal tissue-based research. Therefore, to generate testis-like somatic cells without relying on fetal tissue is of clinical significance. This has previously been attempted by reprogramming human fibroblasts into Sertoli- or Leydig-like cells using a combination of well-chosen transcription factors. Despite expressing a handful of known markers for Sertoli and Leydig cells, the extent to which the in vitro derived cells resemble in vivo cells or recapitulate endogenous functions remain difficult to assess. Furthermore, given the requirement for transgenesis for efficient induction, and for the continuous expression of transcription factors for maintenance of Sertoli and Leydig cell fates, the clinical utility of such cells is unclear.

### SUMMARY OF THE INVENTION

The present invention relates to in vitro methods for production of Sertoli cells and related organoids.

In some preferred embodiments, the present invention provides in vitro methods for production of Sertoli cells from vertebrate pluripotent stem cells comprising: deriving genital ridge cells from pluripotent stem cells: treating the genital ridge cells with a base medium comprising fibroblast growth factor 9 (FGF9), insulin and/or IGF1 so that the genital ridge cells differentiate into Sertoli cells.

In some preferred embodiments, the step of deriving genital ridge cells further comprises: providing vertebrate pluripotent stem cells in a maintenance medium comprising a ROCK inhibitor; at day 0, removing the maintenance medium comprising a ROCK inhibitor and culturing the vertebrate pluripotent stem cells with the base medium comprising CHIR99021 so that the vertebrate pluripotent stem cells differentiate into presomitic mesoderm cells: on about day 4, removing the base medium comprising CHIR99021 and culturing the presomitic mesoderm cells in base medium comprising fibroblast growth factor 9 (FGF9) and heparin so that the presomitic mesoderm cells differentiate into intermediate mesoderm cells: on about day 7, removing the medium comprising FGF9 and heparin and culturing the intermediate mesoderm cells in base medium so that the intermediate mesoderm cells differentiate into genital ridge cells.

In some preferred embodiments, the step of treating the genital ridge cells with a culture medium comprising insulin and/or IGF1 so that the genital ridge cells differentiate into Sertoli cells further comprises on about day 10, removing the base medium and culturing the genital ridge cells in base medium comprising insulin-like growth factor 1 (IGF1) and insulin so that the genital ridge cells differentiate into artificial Sertoli cells.

In some preferred embodiments, step of treating the genital ridge with a base medium comprising fibroblast growth factor 9 (FGF9), insulin and/or IGF1 so that the genital ridge cells differentiate into Sertoli cells further comprises treating the genital ridge cells with epidermal growth factor (EGF), bone morphogenetic protein 4 (BMP4). IWR1, or combinations thereof. In some preferred embodiments, the step of treating the genital ridge with a base medium comprising fibroblast growth factor 9 (FGF9), insulin and/or IGF1 so that the genital ridge cells differentiate into Sertoli cells further comprises treating the genital ridge cells with follicle stimulating hormone (FSH) and/or luteinizing hormone and/or testosterone or a combination thereof.

In some preferred embodiments, the vertebrate pluripotent stem cells are human embryonic stem cells (hESC).

In some preferred embodiments, the present invention provides in vitro methods for production of Sertoli cells from vertebrate pluripotent stem cells comprising: deriving anterior intermediate mesoderm cells from pluripotent stem cells: treating the anterior intermediate mesoderm cells with a base medium comprising insulin and/or IGF1 so that the anterior intermediate mesoderm cells differentiate into Sertoli cells.

In some preferred embodiments, the step of deriving anterior intermediate mesoderm cells further comprises: providing vertebrate pluripotent stem cells in a maintenance medium comprising a ROCK inhibitor: at day 0, removing the maintenance medium comprising a ROCK inhibitor and culturing the vertebrate pluripotent stem cells with the base medium comprising CHIR99021 so that the vertebrate pluripotent stem cells differentiate into presomitic mesoderm cells; and on about day 4, removing the base medium comprising CHIR99021 and culturing the presomitic mesoderm cells in base medium comprising fibroblast growth factor 9 (FGF9) and heparin so that the presomitic mesoderm cells differentiate into intermediate mesoderm cells.

In some preferred embodiments, the step of treating the intermediate mesoderm cells with a culture medium comprising insulin and/or IGF1 so that the intermediate mesoderm cells differentiate into Sertoli cells further comprises on about day 7, removing the medium comprising FGF9 and heparin and culturing the intermediate mesoderm cells in base medium comprising insulin-like growth factor 1 (IGF1) and insulin so that the intermediate mesoderm cells differentiate into artificial Sertoli cells.

In some preferred embodiments, the step of treating the intermediate mesoderm cells further comprises treating the intermediate mesoderm cells with FGF9, epidermal growth factor (EGF), bone morphogenetic protein 4 (BMP4), IWR1, or combinations thereof. In some preferred embodiments, FGF9, epidermal growth factor (EGF), and bone morphogenetic protein 4 (BMP4) are utilized. In some preferred embodiments, FGF9, epidermal growth factor (EGF), bone morphogenetic protein 4 (BMP4), and IWR1 are utilized. In some preferred embodiments, the step of treating the intermediate mesoderm cells further comprises treating the genital ridge cells with follicle stimulating hormone (FSH) and/or luteinizing hormone (LH) and/or testosterone or a combination thereof. In some preferred embodiments, the one or more hormones are added on about day 9.

In some preferred embodiments, the vertebrate pluripotent stem cells are human embryonic stem cells (hESC).

In some preferred embodiments, the step of deriving anterior intermediate mesoderm cells further comprises: providing vertebrate pluripotent stem cells in a maintenance medium comprising LIF; at day 0, removing the maintenance medium comprising LIF and culturing the vertebrate pluripotent stem cells with the base medium comprising Activin A and bFGF so that the vertebrate pluripotent stem cells differentiate into epiblast cells; and on about day 2, removing the base medium comprising Activin A and bFGF and culturing the epiblast cells in base medium comprising Activin A and RA so that the epiblast cells differentiate into intermediate mesoderm cells.

In some preferred embodiments, the step of treating the intermediate mesoderm cells with a culture medium comprising insulin and/or IGF1 so that the intermediate mesoderm cells differentiate into Sertoli cells further comprises on about day 4, removing the medium comprising Activin A and RA and culturing the intermediate mesoderm cells in base medium comprising insulin-like growth factor 1 (IGF1) and insulin so that the intermediate mesoderm cells differentiate into artificial Sertoli cells. In some preferred embodiments, the vertebrate pluripotent stem cells are mouse embryonic stem cells (mESC).

In some preferred embodiments, the artificial Sertoli cells produced by any of the foregoing methods express at least one of the markers selected from the group consisting of EMX2, WT, SOX9, and LHX9. In some preferred embodiments, the artificial Sertoli cells express at least two of the markers selected from the group consisting of EMX2, WT, SOX9, and LHX9. In some preferred embodiments, the artificial Sertoli cells express at least three of the markers selected from the group consisting of EMX2, WT, SOX9, and LHX9. In some preferred embodiments, the artificial Sertoli cells express the markers EMX2, WT, SOX9, and LHX9.

In some preferred embodiments, the base medium utilized in the foregoing methods is an APEL2 medium. In some preferred embodiments, the maintenance medium is a mTESR medium. In some preferred embodiments, the base medium is a DMEM medium.

In some preferred embodiments, the base medium comprising insulin-like growth factor 1 (IGF1) and insulin further in any of the foregoing methods comprises one or more of retinoic acid, PDG₂, and FGF9, and preferably a combination thereof.

In some preferred embodiments, the vertebrate pluripotent stem cells utilized in any of the foregoing methods comprise an exogenous gene.

In some preferred embodiments, the methods described above further comprise the step of culturing the artificial Sertoli cells under conditions such that the artificial Sertoli cells form an artificial Sertoli cell organoid. In some preferred embodiments, the cells are first dissociated. In some preferred embodiments, the dissociated cells are further cultured in an aggrewell plate.

In some preferred embodiments, the artificial Sertoli cell organoid is characterized by having a tubule structure. In some preferred embodiments, the artificial Sertoli cell organoid is further characterized by comprising smooth muscle actin.

In some preferred embodiments, the methods described above further comprise the step of isolating the artificial Sertoli cells or artificial Sertoli cell organoid.

In some preferred embodiments, the methods described above further comprise transplanting the isolated artificial Sertoli cells or artificial Sertoli cell organoid into a mammal.

In some preferred embodiments, the methods described above further comprise contacting the artificial Sertoli cells or artificial Sertoli cell organoid with a test reagent and assaying the effect of the test reagent on the artificial Sertoli cells or artificial Sertoli cell organoid.

In some preferred embodiments, the patient prior to gonadotoxic treatment (chemotherapy and radiation) preserves testicular tissue or somatic cells so that germline stem cells may be produced or isolated in the future. In some preferred embodiments, the germ line stem cells obtained from the tissue can be expanded using the in vitro derived cells of the present invention. In some preferred embodiments, the methods described above further comprise obtaining fibroblast tissue to reprogram to induced pluripotent stem cells from a patient which a can be used to make autologous artificial Sertoli cells. In some preferred embodiments, these differentiated cells can be combined with germline stem cells: either primordial germ cell like cells (PGCLC), prospermatogonia (proSSC), or neonatal/adult spermatogonial stem/progenitor cells (SSC/SPCs). In some preferred embodiments, the stem cells can either proliferate or differentiate into spermatogonia or later germ cell stages. In some preferred embodiments, the methods further comprise transferring the expanded stem cells or differentiated spermatogonia back to a patient in need thereof.

In some preferred embodiments, the present invention provides a cell culture comprising artificial Sertoli cells produced by any of the methods described above.

In some preferred embodiments, the present invention provides isolated artificial Sertoli cells produced by any of the foregoing methods.

In some preferred embodiments, the present invention provides an artificial Sertoli cell organoid produced by any of the methods described above.

In some preferred embodiments, the present invention provides methods comprising: providing artificial Sertoli cells or organoids as described above; contacting the artificial Sertoli cells or organoids with a test reagent; and assaying the effect of the test reagent on the artificial Sertoli cells or organoids.

In some preferred embodiments, the present invention provides methods comprising: providing artificial Sertoli cells or organoids as described above; and transplanting the artificial Sertoli cells or organoids into a subject.

In some preferred embodiments, the present invention provides methods comprising: providing artificial Sertoli cells or organoids as described above: obtaining stem cells or tissue comprising stem cells from a patient; and culturing the stem cells or tissue comprising stem cells from the patient the artificial Sertoli cells. In some preferred embodiments, the stem cells or tissue comprising stem cells are obtained from the patient prior to a gonadotoxic treatment and stored. In some preferred embodiments, the germ line stem cells obtained from the tissue can be expanded using the in vitro derived cells of the present invention. In some preferred embodiments, the methods described above further comprise obtaining fibroblast tissue to reprogram to induced pluripotent stem cells from a patient which a can be used to make autologous artificial Sertoli cells. In some preferred embodiments, these differentiated cells can be combined with germline stem cells: either primordial germ cell like cells (PGCLC), prospermatogonia (proSSC), or neonatal/adult spermatogonial stem/progenitor cells (SSC/SPCs). In some preferred embodiments, the stem cells can either proliferate or differentiate into spermatogonia or later germ cell stages. In some preferred embodiments, the methods further comprise transferring the expanded stem cells or differentiated spermatogonia back to a patient in need thereof.

In some preferred embodiments, the present invention provides kits comprising multiple vessels, wherein at least one vessel contains base medium comprising fibroblast growth factor 9 (FGF9) and heparin, and wherein at least one vessel contains base medium comprising insulin-like growth factor 1 (IGF1) and insulin. In some preferred embodiments, the kits further comprise at least one vessel comprising an inhibitor of ROCK I and/or ROCK II in maintenance medium. In some preferred embodiments, the kits further comprise at least one vessel comprising CHIR99021. In some preferred embodiments, the kits further comprise at least one vessel comprising BMP4. In some preferred embodiments, the kits further comprise at least one vessel comprising EGF. In some preferred embodiments, the kits further comprise at least one vessel comprising IWR1. In some preferred embodiments, the kits further comprise at least one vessel comprising FSH. In some preferred embodiments, the kits further comprise at least one vessel comprising LH. In some preferred embodiments, the kits further comprise at least one vessel comprising testosterone. In some preferred embodiments, the kits further comprise instructions for performing a method as described above.

### BRIEF DESCRIPTION OF THE FIGURES

FIG. 1. Schematic showing developmental history of testis somatic cells and key markers that identify major stages.

FIG. 2. In vitro generation of testis-like somatic cells and resulting organoids. (A) Schematic representation of the experimental method used to generate organoids (B-D) Data from RT-qPCR analysis of various markers along the differentiation time course.

FIG. 3. Photomicrographs showing the expression dynamics of pluripotency and gonadal markers across our mouse ESC differentiation trajectory.

FIG. 4. In vitro derived somatic cells resemble the in vivo embryonic gonadal cells. A) Single cell RNA-seq time-course analysis of in vitro differentiation (days 0, 2, 4, 8) identifies eight clusters, as visualized in UMAP space. Note: shown is one experiment, while in vitro differentiation was successfully completed 9 times. B) Contributions of cells collected at day 0, 2, 4, 8 to the 8 clusters, showing emergence of new cell types in later days. (C). Expression profiles of selected specific and nonspecific (D) gonadal differentiation markers. (E) Correlations between our scRNAseq centroids-by-day with published in vivo cell centroids-by-day. (F) Day-by-cluster cell counts for our data (left) and correlations of our 8 cluster centroids with four of the in vivo cell types (right).

FIG. 5. Schematic showing strategy for combining in vitro derived somatic cells with in vivo derived Oct4-egfp pro-spermatogonia.

FIG. 6. Photomicrographs showing that our in vitro derived somatic cells incorporate pro-spermatogonia and promote their differentiation in vitro. In vitro derived somatic like cells are mixed with purified Oct4+ pro-spermatogonia collected from the testis of PND1-2 Oct4-egfptg/+ mice. During the experiment, a subset of Oct4-egfp+ pro-spermatogonial cells maintain expression of a known undifferentiated spermatogonia marker (known as PLZF), but after 4 days of co-culture, a subset of cells transition into an OCT4-dim or -low state and acquire strong expression of Stra8.

FIG. 7. Improved somatic cell differentiation protocol. A) Schematic of somatic cell generation including the 4 most promising drug combinations. B) Expression of the testis somatic cell markers in all 3 cocktails described in 7A. C) Kidney differentiation markers. D) Granulosa cell marker.

FIG. 8. Photomicrographs showing representative staining patterns for gonadal markers obtained from ALL-GF organoids compared to other culture conditions.

FIG. 9. Graph of data showing that testis like somatic cells derived from Cocktail #3 improve stem cell proliferation at 3 and 5 days of in vitro co-culture. Germ cell numbers are determined by quantifying the number of EGFP+ germline stem cells. The starting number of somatic and germ cells are the same for all combinations.

FIG. 10. Generation of human testis somatic cell-like cells. A) Optimized differentiation schema used for H1 and U6 hESC lines. B) Graphs showing tracking of the relative RNA expression of several cell type-specific markers across the differentiation trajectory.

FIG. 11. Photomicrographs showing that our directed differentiation protocol induces the expression of key proteins markers across the differentiation trajectory. The results are virtually identical for H1 and U6 lines.

FIG. 12. Data showing that ScRNAseq analysis identifies 13 clusters, a subset of which emerge late in culture and resemble known differentiated cells found in vivo in human gonadal tissue.

FIG. 13. Improved differentiation efficiency of human somatic-like cells in the presence of hormone. A) Revised differentiation schema. B) qPCR expression of gonadal, Sertoli. Leydig. and off-target markers.

FIG. 14. Representative images of gonadal markers at day 22 from both H1 ESC lines.

FIG. 15. Photomicrographs showing an organoid consisting of in vitro derived germ cells and somatic cells after 5 days of mixed culture.

FIG. 16. IPSC differentiation schema and graphs presenting data related to validation of gonadal cell generation.

### DEFINITIONS

As used herein the term “stem cell” (“SC”) refers to cells that can self-renew and differentiate into multiple lineages. A stem cell is a developmentally pluripotent or multipotent cell. A stem cell can divide to produce two daughter stem cells, or one daughter stem cell and one progenitor (“transit”) cell, which then proliferates into the tissue's mature, fully formed cells. Stem cells may be derived, for example, from embryonic sources (“embryonic stem cells”) or derived from adult sources. For example, U.S. Pat. No. 5,843,780 to Thompson describes the production of stem cell lines from human embryos. PCT publications WO 00/52145 and WO 01/00650 describe the use of cells from adult humans in a nuclear transfer procedure to produce stem cell lines.

Examples of adult stem cells include, but are not limited to, hematopoietic stem cells, neural stem cells, mesenchymal stem cells, and bone marrow stromal cells. These stem cells have demonstrated the ability to differentiate into a variety of cell types including adipocytes, chondrocytes, osteocytes, myocytes, bone marrow stromal cells, and thymic stroma (mesenchymal stem cells): hepatocytes, vascular cells, and muscle cells (hematopoietic stem cells): myocytes, hepatocytes, and glial cells (bone marrow stromal cells) and, indeed, cells from all three germ layers (adult neural stem cells).

As used herein, the term “totipotent cell” refers to a cell that is able to form a complete embryo (e.g., a blastocyst).

As used herein, the term “pluripotent cell” or “pluripotent stem cell” refers to a cell that has complete differentiation versatility, e.g., the capacity to grow into any of the mammalian body's approximately 260 cell types. A pluripotent cell can be self-renewing and can remain dormant or quiescent within a tissue. Unlike a totipotent cell (e.g., a fertilized, diploid egg cell), a pluripotent cell, even a pluripotent embryonic stem cell, cannot usually form a new blastocyst.

As used herein, the term “induced pluripotent stem cells” (“iPSCs”) refers to a stem cell induced from a somatic cell, e.g., a differentiated somatic cell, and that has a higher potency than said somatic cell. iPS cells are capable of self-renewal and differentiation into mature cells.

As used herein, the term “multipotent cell” refers to a cell that has the capacity to grow into a subset of the mammalian body's approximately 260 cell types. Unlike a pluripotent cell, a multipotent cell does not have the capacity to form all of the cell types.

As used herein, the term “progenitor cell” refers to a cell that is committed to differentiate into a specific type of cell or to form a specific type of tissue.

As used herein, the term “embryonic stem cell” (“ES cell” or ESC”) refers to a pluripotent cell that is derived from the inner cell mass of a blastocyst (e.g., a 4- to 5-day-old human embryo) and has the ability to yield many or all of the cell types present in a mature animal.

As used herein the term “feeder cells” refers to cells used as a growth support in some tissue culture systems. Feeder cells may be embryonic striatum cells or stromal cells. As used herein, the term “chemically defined media” refers to culture media of known or essentially-known chemical composition, both quantitatively and qualitatively. Chemically defined media is free of all animal products, including serum or serum-derived components (e.g., albumin).

As used herein, the term “serum-free media” refers to culture media that is devoid of serum, but not necessarily of other undefined components.

### DETAILED DESCRIPTION OF THE INVENTION

Methods, kits, compositions, and systems are provided for culturing pluripotent stem cells to produce populations of cells comprising artificial Sertoli cells. In particular, culture conditions are provided that result in the generation of artificial Sertoli cells from a starting culture of human pluripotent stem cells.

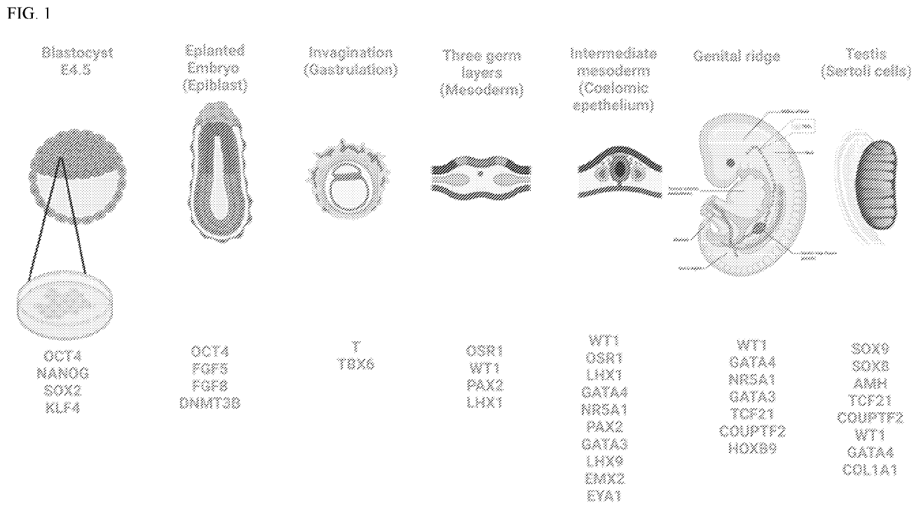

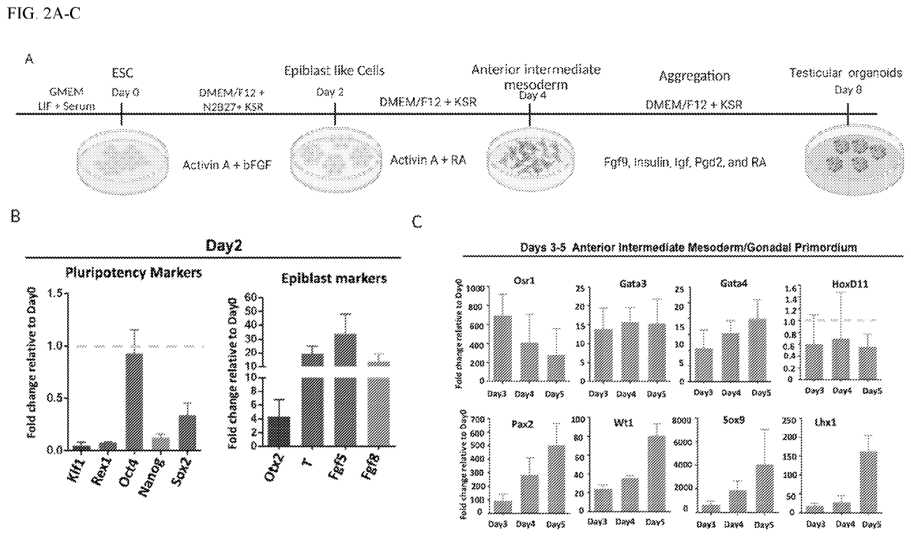

These methods overcome the limitations noted in the Background and leverage genetic, evolutionary, and molecular insights gained from scRNAseq data that the inventors collected across developmental stages and multiple species to develop a new, highly efficient, directed somatic cell differentiation protocol. The inventors have tuned many parameters of the protocols on a mouse ESC and two human ESC lines and analysed scRNAseq data to confirm the progression through expected cell states along the developmental trajectories. Benchmarking and classification of the in vitro derived cell states has relied on the wealth of in vivo markers that have been previously described during gonadal differentiation (see FIG. 1).

Somatic cells of the testis are central to testis tissue homeostasis and men's reproductive and overall health. The somatic cells provide a series of unknown growth factors and cytokines that are necessary for guiding germ cell development in vivo and required for the complete reconstitution of germline development for females in vitro or promoting the differentiation of male primordial germ cell like cells (PGCLC) to spermatogonia.

When working with mice, co-culturing of in vitro derived PGCLC with fetal somatic cells isolated from littermates or allogenic embryonic gonads is feasible, but this is costly in non-human primates and ethically inconceivable in human. Thus, an improved understanding of the somatic cell specification program and generation of alternative somatic cell sources will prove critical for reconstituting somatic cells in a petri-dish, replacement of damaged cells in vivo in response to inherited/natural occurring genetic mutations, iatrogenic agents because of cancer therapy, or to rejuvenate the aging gonads. To address this gap, the present inventors utilized single cell RNAseq data to provide a method for the generation of human artificial Sertoli cells from ESCs using the differentiation schema described below.

### Pluripotent Stem Cells

The methods systems, systems and kit of the present invention find use with a variety of pluripotent cells. Suitable pluripotent stem cells include, but are not limited to, embryonic stem cells, adult stem cells, and induced pluripotent stem cells. In some preferred embodiments, the pluripotent stem cells are vertebrate pluripotent stem cells. In some particularly preferred embodiments, the pluripotent stem cells are human embryonic stem cells (hESCs). In other particularly preferred embodiments, the pluripotent stem cells are mouse embryonic stem cells (mESCs).

In some preferred embodiments, the pluripotent stem cells may be genetically modified by methods known in the art so that they comprise and express one or more exogenous genes.

### Base Media

A concern in the culture of human ES cells is to remove, to the extent possible, undefined constituents and constituents of animal origin from ES cell culture conditions. Standardizing culture conditions minimizes the normal variations in biological materials to which the cells are exposed. Further, by avoiding the use of materials, cells, exudates or constituents of animal origin, one can avoid possible cross-species viral transmission through the culture system. Thus, utilization of chemically defined media (CDM) that avoid the use of animal products provides a baseline culture condition upon which differentiation factors may be added with predictable effects.

CDM (e.g., for hESCs) may include maintenance or basal media containing salts, vitamins, glucose and amino acids. For maintenance of human stem cells prior to the differentiation protocol, a mTeSR medium such as mTeSR1™ from StemCell Technologies may be utilized. In some embodiments, the maintenance medium preferably comprises a ROCK inhibitor such as Y27632. Maintenance medium for mouse stem cells may preferably be GMEM from ThermoFisher Scientific, preferably supplemented with LIF (Leukemia Inhibitory Factor) and optionally knockout serum. The basal differentiation medium can be any of a number of commercially available media. In some preferred embodiments, a combination of Dulbecco's Modified Eagle Medium and Hams F12 medium, sold as a combination (DMEM/F12: Invitrogen) may be utilized. In other preferred embodiments, an APEL medium may be utilized, for example, STEMdiff™ APEL™ medium from StemCell Technologies. STEMdiff™ APEL™ Medium is a serum-free and animal component-free medium specifically developed to support hPSC differentiation. It was first described for the induction of hemato-endothelial cells, when supplemented with VEGF, BMP-4, SCF, and Activin A, but it has also been proven to be an effective basal medium for hPSC differentiation to other lineages, including cardiomyocytes. In other preferred embodiments, an mTeSR medium may be utilized for maintenance of stem cells.

Differentiation into Artificial Sertoli Cells

The present invention provides methods and reagents for producing artificial Sertoli cells from pluripotent stems cells. The present invention is not limited to the use of any particular pluripotent stem cells or chemically defined media. The methods described herein for the production of artificial Sertoli cells are described in relation to events occurring at various time points. It will be recognized that the methods may be varied by making alterations to the described time schedules. “Day 0” as used herein refers to the day and time that the pluripotent stem cells are removed from a maintenance medium and exposed to a differentiation medium. The differentiation timeline is then defined from the Day 0 starting point. When the term “about” X days is utilized, it refers to the number of days from the Day 0 starting time point plus or minus 12 hours. For example, “on about Day 4” means 96 hours (i.e., 4 days) from the Day 0 starting point plus or minus 12 hours. If the Day 0 stating time was 9:00 AM, “about Day 4” then refers to 96 hours from that time point plus or minus 12 hours.

The first step in a method for producing human artificial Sertoli cells according to the invention comprises providing pluripotent hESC as described above. In some preferred embodiments, the pluripotent stem cells are provided in a stem cell maintenance medium. In some preferred embodiments, the stem cell maintenance medium is a chemically defined medium such as an mTeSR medium. In some preferred embodiments, the stem cell maintenance medium comprises a ROCK inhibitor. In some preferred embodiments, the ROCK inhibitor is Y27632.

The second step of the method of the present invention comprises removing the pluripotent stem cells from the maintenance medium and culturing the pluripotent stem cells in a basal medium supplemented with agents suitable for directing the pluripotent stem cells to a presomitic mesoderm lineage. In some preferred embodiments, the basal medium is a chemically defined medium. In some particularly preferred embodiments, the basal medium is an APEL medium such as STEMdiff™ APEL™ medium from StemCell Technologies. In some particularly preferred embodiments, the basal medium is supplemented with from between 0.5 to 15 μM (1.0, 1.5, 2.0, 2.5, 3.0, 3.5, 4.0, 5.0, 6.0, 7.0, 8.0, 9.0, 10.0, 11.0, 12.0, 13.0, and 14.0 μM and values and ranges therein) CHIR99021. This step defines Day 0 of the process. The base medium with supplements is preferably changed daily.

The third step of the method of the present invention comprises on about or at day 4 culturing the presomitic mesoderm cells produced in step 2 in a basal medium supplemented with agents for directing the presomitic mesoderm cells to form intermediate mesoderm. In some preferred embodiments, the basal medium is a chemically defined medium. In some particularly preferred embodiments, the basal medium is an APEL medium such as STEMdiff™ APEL™ medium from StemCell Technologies. In some particularly preferred embodiments, the basal medium is supplemented with from between 20 and 500 ng/ml (50, 100, 150, 200, 250, 300, 350, 400, 450 ng/ml and values and ranges therein) FGF 9. In some particularly preferred embodiments, the basal medium is further supplemented with from between 0.1 and 10 μg/ml (0.4, 0.8, 1.0, 1.5, 2.0, 3.0, 5.0, 6.0, 7.0, 8.0, 9.0 μg/ml and values and ranges therein) heparin. The base medium with supplements may preferably be changed every two days.

The fourth step of the method of the present invention comprises on about or at day 7 culturing the intermediate mesoderm cells produced in step 3 in a basal medium to direct the intermediate mesoderm cells to form genital ridge cells. In some preferred embodiments, the basal medium is a chemically defined medium. In some particularly preferred embodiments, the basal medium is an APEL medium such as STEMdiff™ APEL™ medium from StemCell Technologies. In some particularly preferred embodiments, the basal medium is not supplemented with additional differentiation agents during this step. The base medium may preferably be changed every two days.

Optionally, the fourth step may be skipped, and the intermediate mesoderm cells may be cultured in the medium described in Step 5 on or about day 7.

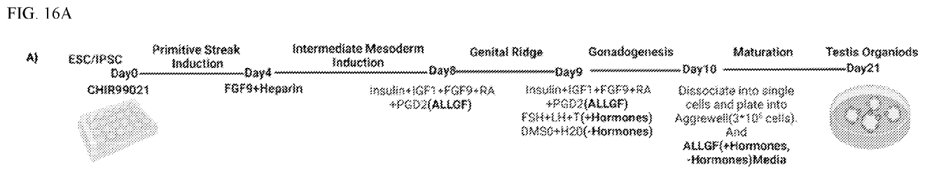

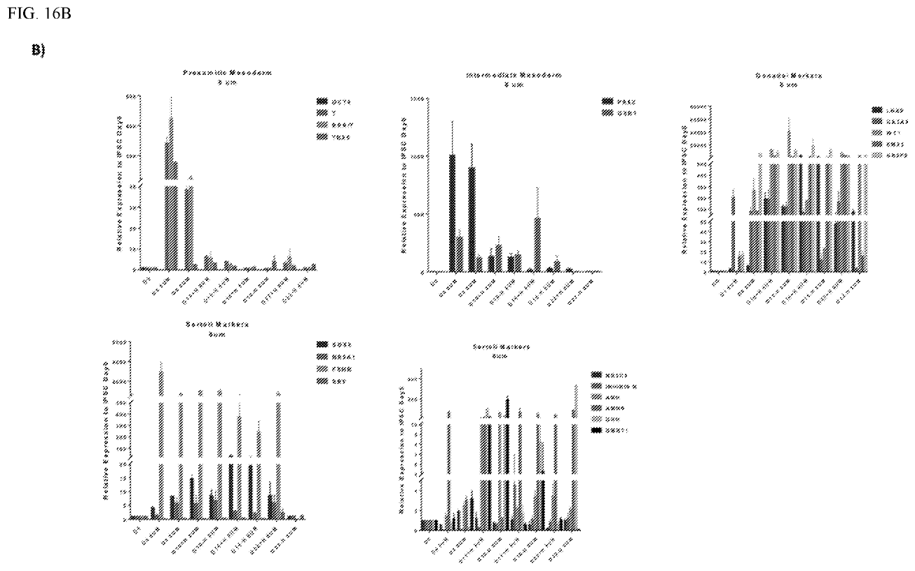

The fifth step of the method of the present invention comprises on about or at day 10 (or about day 7 if the fourth step is skipped, see FIG. 16A) culturing the genital ridge cells produced in step 4 in a basal medium supplemented with agents for directing the genital ridge cells to form artificial Sertoli cells and other gonadal progenitors. In some preferred embodiments, the basal medium is a chemically defined medium. In some particularly preferred embodiments, the basal medium is an APEL medium such as STEMdiff™ APEL™ medium from StemCell Technologies. In some particularly preferred embodiments, the basal medium is supplemented with from between 5 and 100 nM (10, 17, 20, 30, 40, 50, 60, 70, 80, 90 nM and values and ranges therein) IGF1. In some particularly preferred embodiments, the basal medium is further supplemented with from between 10 and 500 nM (20, 50, 100, 200, 300, 400 nM and values and ranges therein) insulin. It is contemplated that the inclusion of IGF1 and insulin in the base medium is sufficient to drive differentiation of the genital ridge cells to artificial Sertoli cells.

However, in other additional embodiments, the base medium may be supplemented with one or more other agents. In some embodiments, the base medium may be further supplemented with from between 0.01 and 10 μM (0.05, 0.1, 1.0, 5.0, 8.0 UM and values and ranges therein) retinoic acid (RA). In some embodiments, the base medium may be further supplemented with from between 50 and 1000 ng/ml (100, 200, 300, 400, 500, 600, 700, 800, 900 and values and ranges therein) PGD₂. In some embodiments, the base medium may be further supplemented with from between 50 and 500 ng/ml (100, 200, 300, 400 and values and ranges therein) FGF9. In some further preferred embodiments, the base medium used in step 5 may be further be supplemented with from 5 to 100 mg/ml (10, 20, 30, 40, 50, 60, 70, 80, 90 and values and ranges therein) bone morphogenetic protein 4 (BMP4). In some further preferred embodiments, the base medium used in step 5 may be further be supplemented with from 10 to 200 mg/ml (30, 50, 70, 100, 150 and values and ranges therein) epidermal growth factor (EGF). In some still further preferred embodiments, the base medium used in step 5 may be further be supplemented with from 0.5 to 10 μM (1.0, 2.0, 3.0, 4.0 5.0, 6.0, 7.0, 8.0, 9.0 μM and values and ranges therein) IWR1. In some preferred embodiments, a combination of insulin and IGF1 in the above ranges is utilized. In some preferred embodiments, a combination of insulin, IGF1, and FGF9) in the above ranges is utilized. In some preferred embodiments, a combination of insulin. IGF1. FGF9, RA and PGD₂ in the above ranges is utilized. In some preferred embodiments, a combination of insulin, IGF1, FGF9, BMP4, EGF and/or IWR1 in the above ranges is utilized. In some preferred embodiments, a combination of insulin, IGF1, FGF9, BMP4, EGF, RA, IWR1, and PGD₂ in the above ranges is utilized. The base medium with supplements may preferably be changed every two days.

In some further preferred embodiments, the base medium used in step 5 is further supplemented with one or more hormones. In some further preferred embodiments, the one or more hormones are added at about day 9. In some preferred embodiments, the base medium (which may preferably comprise one or more of insulin, IGF1, FGF9, BMP4, EGF, RA, IWR1 and PGD₂ in the above ranges) is further supplemented with from 5 to 100 mg/ml (10, 20, 30, 40, 50, 60, 70, 80, 90 and values and ranges therein) luteinizing hormone (LH). In some preferred embodiments, the base medium (which may preferably comprise one or more of insulin, IGF1, FGF9, BMP4, EGF, RA, IWR1 and PGD₂ in the above ranges) is further supplemented with from 20 to 300 mg/ml (50, 100, 150, 200, 250 and values and ranges therein) follicle stimulating hormone (FSH). In some preferred embodiments, the base medium (which may preferably comprise one or more of insulin, IGF1, FGF9, BMP4, EGF, RA. IWR1, and PGD₂ in the above ranges) is further supplemented with from between 0.01 and 10 μM (0.05, 0.1, 1.0, 5.0, 8.0 μM and values and ranges therein) testosterone. In some preferred embodiments, the base medium (which may preferably comprise one or more of insulin, IGF1, FGF9, BMP4, EGF, RA, IWR1 and PGD₂ in the above ranges) with LH. FSH and testosterone in the ranges described herein.

In some preferred embodiments, the cultures of step 5 for hESC are maintained until artificial Sertoli cells and/or organoids are derived or until about day 13 and most preferably until about days 18 to 22. In some preferred embodiments, a sixth step of the methods of the present invention comprises dissociation of the differentiated cells. In some preferred embodiments, the differentiated cells are harvested for dissociation at from about day 10 to day 20, preferably about from day 15 to day 18, and most preferably on or about day 18. In some preferred embodiments, the cells are plated into an aggrewell (Aggrewell 400, 24 well plate (Stemcell Technologies Cat #34421)) and the organoids allowed to form. In some preferred embodiments, the organoids are collected on or about days 20 to 24, and most preferably on days 20 to 22.

The first step in a method for producing mouse artificial Sertoli cells according to the invention comprises providing pluripotent mESC as described above. In some preferred embodiments, the pluripotent stem cells are provided in a stem cell maintenance medium. In some preferred embodiments, the stem cell maintenance medium is GMEM supplemented with LIF and serum.

The second step of the method of the present invention comprises removing the pluripotent stem cells from the maintenance medium and culturing the pluripotent stem cells in a basal medium supplemented with agents suitable for directing the pluripotent stem cells to epiblast. This is defined as Day 0. In some preferred embodiments, the basal medium is DMEM/F12 and Neurobasal Medium (both from ThermoFisher Scientific) in a ratio of from 2:1 to 1:2 and most preferably at a ratio of about 1:1. In some preferred embodiments, the basal medium is supplemented with N2 supplement, B-27 supplement, and Knockout Serum Replacer (KSR: all from ThermoFisher Scientific), Activin A and bFGF. The supplemented basal medium is referred to as priming medium. In some particularly preferred embodiments, the basal medium is supplemented with from 1 to 10 μl/ml (1.0, 2.0, 3.0, 4.0, 5.0, 6.0, 7.0, 8.0, 9.0, 10.0 μl/ml and ranges and values therein) N2 supplement. In some particularly preferred embodiments, the basal medium is supplemented with from 1 to 20 μl/ml (1.0, 5.0, 10.0, 20.0 μl/ml and ranges and values therein) B-27 supplement. In some particularly preferred embodiments, the basal medium is supplemented with from 1 to 20 μl/ml (1.0, 5.0, 10.0, 20.0 μl/ml and ranges and values therein) KSR. In some particularly preferred embodiments, the basal medium is supplemented with from 1 to 20 ng/ml (1.0, 5.0, 10.0, 20.0 ng/ml and ranges and values therein) Activin A. In some particularly preferred embodiments, the basal medium is supplemented with from 1 to 20 ng/ml (1.0, 5.0, 10.0, 20.0 ng/ml and ranges and values therein) bFGF.

The third step of the method of the present invention comprises removing the epiblasts from the priming medium used in step 2 at about day 2 and culturing the epiblasts in a basal medium supplemented with agents suitable for directing the epiblast cells to form anterior intermediate mesoderm. In some preferred embodiments, the basal medium is DMEM/F12. In some preferred embodiments, the basal medium is supplemented with KSR, Activin A and Retinoic Acid (RA). The supplemented basal medium is referred to as differentiation medium. In some particularly preferred embodiments, the basal medium is supplemented with from 1 to 20 μl/ml (1.0, 4.0, 5.0, 10.0, 20.0 μl/ml and ranges and values therein) KSR. In some particularly preferred embodiments, the basal medium is supplemented with from 1 to 20 ng/ml (1.0, 5.0, 10.0, 20.0 ng/ml and ranges and values therein) Activin A. In some particularly preferred embodiments, the basal medium is supplemented with from 10 to 200 nM (10.0, 50.0, 100.0, 200.0 nM) and ranges and values therein) RA.

The fourth step of the method of the present invention comprises removing the anterior intermediate mesoderm cells from the differentiation medium used in step 3 on about day 4 and culturing the anterior intermediate mesoderm cells in a basal medium supplemented with agents suitable for directing the anterior intermediate mesoderm cells to form Sertoli cells and/or testis organoids comprising artificial Sertoli cells. In some preferred embodiments, the basal medium is DMEM/F12. In some preferred embodiments, the basal medium is supplemented with KSR, FGF9, IGF1, insulin, PDG2 and RA. The supplemented medium may be referred to as aggregation medium. In some particularly preferred embodiments, the basal medium is supplemented with from 1 to 20 μl/ml (1.0, 4.0, 5.0, 10.0, 20.0 μl/ml and ranges and values therein) KSR. In some particularly preferred embodiments, the basal medium is supplemented with from between 5 and 100 nM (10, 17, 20, 30, 40, 50, 60, 70, 80, 90 nM and values and ranges therein) IGF1. In some particularly preferred embodiments, the basal medium is further supplemented with from between 10 and 500 nM (20, 50, 100, 200, 300, 400 nm and values and ranges therein) insulin. It is contemplated that the inclusion of IGF1 and insulin in the base medium is sufficient to drive differentiation of the anterior intermediate mesoderm cells to form artificial Sertoli cells and organoids comprising the cells. However, in other additional embodiments, the base medium may be supplemented with one or more other agents. In some embodiments, the base medium may be further supplemented with from between 10.0 and 200 nM (10.0, 20.0, 50.0, 100.0 and 200.0 nM and values and ranges therein) RA. In some embodiments, the base medium may be further supplemented with from between 50 and 1000 ng/ml (100, 200, 300, 400, 500, 600, 700, 800, 900 ng/ml and values and ranges therein) PGD₂. In some embodiments, the base medium may be further supplemented with from between 50 and 500 ng/ml (100, 200, 300, 400 and values and ranges therein) FGF9. In some preferred embodiments, the base medium may be further supplemented with from between 1 and 10 μM (1.0, 2.0, 3.0, 4.0, 5.0, 6.0, 7.0, 8.0, 9.0, 10.0 μM and ranges and values therein) Y-27632. In some further preferred embodiments, the base medium used in step 5 may be further be supplemented with from 5 to 100 mg/ml (10, 20, 30, 40, 50, 60, 70, 80, 90 and values and ranges therein) bone morphogenetic protein 4 (BMP4). In some further preferred embodiments, the base medium used in step 5 may be further be supplemented with from 10 to 200 mg/ml (30, 50, 70, 100, 150 and values and ranges therein) epidermal growth factor (EGF). In some further preferred embodiments, the base medium used in step 5 may be further be supplemented with from 0.5 to 10 μM (0.5, 1, 2, 4, 5, 7, 10 and values and ranges therein) of IWR1. The base medium with supplements may preferably be changed every two days.

In some preferred embodiments, the cultures of step 4 for mESC are maintained until artificial Sertoli cells and/or organoids are derived or until about day 8. In some preferred embodiments, the methods of the present invention comprise dissociation of the differentiated mESC cells after step 4. In some preferred embodiments, the cells are plated into an aggrewell (Aggrewell 400, 24 well plate (Stemcell Technologies Cat #34421)) and the organoids allowed to form. In some preferred embodiments, the organoids are collected on or about days 6 to 10, and most preferably on about day 8.

In some preferred embodiments, the artificial Sertoli cells produced by the methods described above express at least one of the markers selected from the group consisting of EMX2, WT, SOX9, and LHX9. In some preferred embodiments, the artificial Sertoli cells express at least two of the markers selected from the group consisting of EMX2, WT, SOX9, and LHX9. In some preferred embodiments, the artificial Sertoli cells express at least three of the markers selected from the group consisting of EMX2, WT, SOX9, and LHX9. In some preferred embodiments, the artificial Sertoli cells express the markers EMX2, WT, SOX9, and LHX9.

In some preferred embodiments, the cultured are maintained until artificial Sertoli cells form organoids. The artificial Sertoli organoids preferably are three dimensional organoids having a roughly spherical shape. In some preferred embodiments, the organoids are characterized by comprising tubule structures. In some preferred embodiments, the organoids are characterized by comprising smooth muscle actin.

In some preferred embodiments, the artificial Sertoli cells or organoids may be harvested or isolated from the cultures for further use.

### Uses of Artificial Sertoli Cell and Organoids

In some preferred embodiments, methods, reagents, and kits described herein, as well as the artificial Sertoli cells and organoids generated therewith, find use in various research, diagnostic, clinical, and therapeutic applications. In some embodiments, artificial Sertoli cells or organoids are used for direct transplantation into a subject (e.g., for the treatment of infertility, etc.). In some embodiments, artificial Sertoli cells generated by methods herein are useful for diagnostic, prognostic, and/or therapeutic uses.

In some embodiments, the isolated artificial Sertoli cells or organoids may be directly transplanted in a subject. If appropriate, cells are co-administered with one or more pharmaceutical agents or bioactives that facilitate the survival and function of the transplanted cells.

In some embodiments, human organoids are transplanted into mice for additional differentiation and/or maturation of the cells in the organoids. In other embodiments, the organoids are preferably combined with in vivo or in vitro derived germ cells to achieve human germline stem cell expansion and further promote differentiation. It is contemplated that these methods will result in production of haploid round or elongating spermatids which find use in assisted reproductive technologies such as IVF/ICSI.

In some embodiments, the Sertoli cells or organoids of the invention may be used to co-culture gamete stem cells such as primordial germ cell like cells, prospermatogonia or spermatogonial stem/progenitor cells (SSC/SPCs) from a patient. In some preferred embodiments, the cells are cultured so that the stem cells from the patient differentiate into spermatogonia. In some preferred embodiments, the gamete stem cells or cells derived from the gamete stem cells such as spermatogonia are transplanted back into the patient or a patient in need thereof. In some preferred embodiments, the patient has previously undergone a gonadotoxic treatment, including but not limited to chemotherapy and/or radiation. In some preferred embodiments, the stem cells or tissue comprising stem cells are obtained from the patient prior to a gonadotoxic treatment. In some preferred embodiments, prior to gonadotoxic treatment (chemotherapy and radiation), the testicular tissue or somatic cells of the subject are preserved so that germline stem cells may be produced or isolated in the future. In some preferred embodiments, the germ line stem cells obtained from the tissue can be expanded using the in vitro derived cells of the present invention. In some preferred embodiments, the methods described above further comprise obtaining fibroblast tissue to reprogram to induced pluripotent stem cells from a patient which a can be used to make autologous artificial Sertoli cells. In some preferred embodiments, these differentiated cells can be combined with germline stem cells: either primordial germ cell like cells (PGCLC), prospermatogonia (proSSC), or neonatal/adult spermatogonial stem/progenitor cells (SSC/SPCs). In some preferred embodiments, the stem cells can either proliferate or differentiate into spermatogonia or later germ cell stages. In some preferred embodiments, the methods further comprise transferring the expanded stem cells or differentiated spermatogonia back to a patient in need thereof.

In some embodiments, the artificial Sertoli cells or organoids may be provided on a support material. Support materials suitable for use for purposes of the present invention include tissue templates, conduits, barriers, and reservoirs useful for tissue repair. In particular, synthetic and natural materials in the form of foams, sponges, gels, hydrogels, textiles, and nonwoven structures, which have been used in vitro and in vivo to reconstruct or regenerate biological tissue, as well as to deliver chemotactic agents for inducing tissue growth, are suitable for use in practicing the methods of the present invention. See, for example, the materials disclosed in U.S. Pat. Nos. 5,770,417, 6,022,743, 5,567,612, 5,759,830, 6,626,950, 6,534,084, 6,306,424, 6,365,149, 6,599,323, 6,656,488, U.S. Published Application 2004/0062753 A1, U.S. Pat. Nos. 4,557,264 and 6,333,029.

Cells generated with methods and reagents herein may be implanted as dispersed cells or formed into implantable clusters. In some embodiments, cells are provided in biocompatible degradable polymeric supports; porous, permeable, or semi-permeable non-degradable devices: or encapsulated (e.g., to protect implanted cells from host immune response, etc.). Cells may be implanted into an appropriate site in a recipient. Suitable implantation sites may include, for example, the testes or subcutaneously.

In some embodiments, cells or cell clusters are encapsulated for transplantation into a subject. Encapsulation techniques are generally classified as microencapsulation, involving small spherical vehicles, and macroencapsulation, involving larger flat-sheet and hollow-fiber membranes (Uludag, H. et al. Technology of mammalian cell encapsulation. Adv Drug Deliv Rev. 2000; 42: 29-64, herein incorporated by reference in its entirety). Methods of preparing microcapsules include those disclosed by Lu M Z, et al. Biotechnol Bioeng. 2000, 70: 479-83; Chang T M and Prakash S, Mol Biotechnol. 2001, 17: 249-60; and Lu M Z. et al, J. Microencapsul. 2000, 17: 245-51.; herein incorporated by reference in their entireties. For example, microcapsules may be prepared by complexing modified collagen with a ter-polymer shell of 2-hydroxy ethyl methylacrylate (HEMA), methacrylic acid (MAA) and methyl methacrylate (MMA), resulting in a capsule thickness of 2-5 μm. Such microcapsules can be further encapsulated with additional 2-5μιη ter-polymer shells in order to impart a negatively charged smooth surface and to minimize plasma protein absorption (Chia, S. M. et al. Multi-layered microcapsules for cell encapsulation Biomaterials. 2002 23: 849-56; herein incorporated by reference in its entirety). In some embodiments, microcapsules are based on alginate, a marine polysaccharide (Sambanis, Diabetes Technol. Ther. 2003, 5: 665-8; herein incorporated by reference in its entirety) or its derivatives. For example, microcapsules can be prepared by the polyelectrolyte complexation between the polyanions sodium alginate and sodium cellulose sulphate with the polycation poly(methylene-co-guanidine) hydrochloride in the presence of calcium chloride.

In some embodiments, cells generated using methods and reagents described herein are microencapsulated for transplantation into a subject (e.g., to prevent immune destruction of the cells). Microencapsulation of cells (e.g., pancreatic lineage cells, beta-like cells, etc.) provides local protection of implanted/transplanted cells from immune attack (e.g., along with or without the use of systemic immune suppressive drugs). In some embodiments, cells and/or cell clusters are microencapsulated in a polymeric, hydrogel, or other suitable material, including but not limited to: poly(orthoesters), poly(anhydrides), poly(phosphoesters), poly(phosphazenes), polysaccharides, polyesters, poly(lactic acid), poly(L-lysine), poly(glycolic acid), poly(lactic-co-glycolic acid), poly(lactic acid-co-lysine), poly(lactic acid-graft-lysine), polyanhydrides, poly(fatty acid dimer), poly(fumaric acid), poly(sebacic acid), poly(carboxyphenoxy propane), poly(carboxyphenoxy hexane), poly(anhydride-co-imides), poly(amides), poly(ortho esters), poly(iminocarbonates), poly(urethanes), poly(organophasphazenes), poly(phosphates), poly(ethylene vinyl acetate), poly(caprolactone), poly(carbonates), poly(amino acids), poly(acrylates), polyacetals, poly(cyanoacrylates), poly(styrenes), poly(vinyl chloride), poly(vinyl fluoride), poly(vinyl imidazole), chlorosulfonated polyolefins, polyethylene oxide, polystyrene, polysaccharides, alginate, hydroxypropyl cellulose (HPC), N-isopropylacrylamide (NIP A), polyethylene glycol, polyvinyl alcohol (PVA), polyethylenimine, chitosan (CS), chitin, dextran sulfate, heparin, chondroitin sulfate, gelatin, etc., and their derivatives, co-polymers, and mixtures thereof. In some embodiments, cell are microencapsulated in an encapsulant comprising or consisting of alginate. Cells may be embedded in a material or within a particle (e.g., nanoparticle, microparticle, etc.) or other structure (e.g., matrix, nanotube, vesicle, globule, etc.). In some embodiments, microencapsulating structures are modified with immune-modulating or immunosuppressive compounds to reduce or prevent immune response to encapsulated cells. For example, pancreatic lineage cells are encapsulated within an encapsulant material (e.g., alginate hydrogel) that has been modified by attachment of an immune-modulating agent (e.g., the immune modulating chemokine, CXCL12 (also known as SDF-1). In some embodiments, such an immune modulating agent is a T-cell chemorepellent and/or a pro-survival factor.

In some embodiments, cells generated using methods and reagents described herein are macroencapsulated for transplantation into a subject. Macroencapsulation of cells, for example, within a permeable or semipermeable chamber, provides local protection of implanted/transplanted cells from immune attack (e.g., along with or without the use of systemic immune suppressive drugs), prevents spread of cells to other tissues or areas of the body, and/or allows for efficient removal of cells. Suitable devices for macroencapsulation include those described in, for example, U.S. Pat. No. 5,914,262: Uludag, et al, Advanced Drug Delivery Reviews, 2000, pp. 29-64, vol. 42, herein incorporated by reference in their entireties.

Other encapsulation (micro or macro) devices and methods may find use in embodiments described herein. For example, methods and devices described in U.S. Pub No. 20130209421, U.S. Pat. No. 8,785,185, each of which are herein incorporated by reference in their entireties, are within the scope of embodiments described herein.

In some embodiments, the Sertoli cells or organoids of the present invention may be used for hormone therapy. In some preferred embodiments, the organoids are encapsulated and subcutaneously implanted in the subject.

In some embodiments, the Sertoli cells or organoids of the present invention may be used for fertility restoration. In some preferred embodiments, endogenous defective somatic cells in the testis are combined or replaced by the Sertoli cells or organoids of the present invention. In other preferred embodiments, the organoids are transplanted into a subject to allow spermatogenesis to occur at an ectopic location (e.g., a subcutaneous location) other than the testis.

In further embodiments, populations of artificial Sertoli cells and organoids may be used to prepare antibodies and cDNA libraries that are specific for the differentiated phenotype. General techniques used in raising, purifying and modifying antibodies, and their use in immunoassays and immunoisolation methods are described in Handbook of Experimental Immunology (Weir & Blackwell, eds.): Current Protocols in Immunology (Coligan et al, eds.); and Methods of Immunological Analysis (Masseyeff et al, eds., Weinheim: VCH Verlags GmbH). General techniques involved in preparation of mR A and cDNA libraries are described in R A Methodologies: A Laboratory Guide for Isolation and Characterization (R. E. Farrell, Academic Press, 1998): cDNA Library Protocols (Cowell & Austin, eds., Humana Press); and Functional Genomics (Hunt & Livesey, eds., 2000). Relatively homogeneous cell populations are particularly suited for use in drug screening and therapeutic applications.

In some embodiments, the artificial Sertoli cells and organoids generated by methods provided herein are used to screen for agents (e.g., small molecule drugs, peptides, polynucleotides, and the like) or environmental conditions (such as culture conditions or manipulation) that affect the cells. Particular screening applications relate to the testing of pharmaceutical compounds in drug research and to agents for use in cryopreservation of gametes including sperm. Assessment of the activity of candidate pharmaceutical compounds generally involves combining the cells with the candidate compound, determining any change in the morphology, marker phenotype, or metabolic activity of the cells that is attributable to the compound (compared with untreated cells or cells treated with an inert compound), and then correlating the effect of the compound with the observed change. Any suitable assays for detecting changes associated with test agents may find use in such embodiments. The screening may be done, for example, either because the compound is designed to have a pharmacological effect on Sertoli cell types, because a compound designed to have effects elsewhere may have unintended side effects, or because the compound is part of a library screen for a desired effect. Two or more drugs can be tested in combination (by combining with the cells either simultaneously or sequentially), to detect possible drug-drug interaction effects. In some applications, compounds are screened for cytotoxicity.

In some embodiments, methods and systems are provided for assessing the safety and efficacy of drugs that act upon Sertoli cells, or drugs that might be used for another purpose but may have unintended effects upon Sertoli cells. In some embodiments, cells described herein find use in high throughput screening (HTS) applications. In some embodiments, a HTS screening platform is provided (e.g., cells and plates) that allows for the rapid testing of large number (e.g., 1×10³, 1×10⁴, 1×10⁵, 1×10⁶ or more) of agents (e.g., small molecule compounds, peptides, etc.). In some embodiments, artificial Sertoli cells or organoids generated using methods and reagents described herein are utilized for therapeutic delivery to a subject. Cells may be placed directly in contact with subject tissue or may be otherwise sealed or encapsulated (e.g., to avoid direct contact). In embodiments in which cells are encapsulated, exchange of nutrients, gases, etc. between the encapsulated cells and the subject tissue is allowed. In some embodiments, cells are implanted/transplanted on a matrix or other delivery platform.

In some embodiments, the methods and kits described herein are useful for identifying additional factors, reagents, and methods for the generation of artificial Sertoli cells or other cell types. The methods used herein may be used to screen factors, reagents and/or conditions for their effect of differentiation. In some embodiments, any screening performed in this or other embodiments discussed herein may be high-throughput screening.

### EXPERIMENTAL

### Example 1

The following example provides a description of the reagents and protocols for producing artificial Sertoli cells according to the present invention from mouse Embryonic Stem Cells (mESC).

Growing mESCs

### Reagents/Materials

1—Mouse embryonic stem cells (TG2A)
2—GMEM (ThermoFisher scientific Cat #1171035)
3—Gelatin solution Type B (Sigma Aldrich Cat #G1393-100ML)
4—MEM Non-Essential Amino Acids (ThermoFisher scientific Cat #11140050)
5—2-Mercaptoethanol (ThermoFisher scientific Cat #21985023)
6—Sodium pyruvate solution (Sigma Aldrich Cat #S8636-100ML)
7—Embryonic stem-cell Fetal bovine serum, qualified (ThermoFisher scientific Cat #16141079)
8—TrypLE Express Enzyme (ThermoFisher scientific Cat #12604013)
9—Leukemia Inhibitory Factor (Sigma Aldrich Cat #L5283-10 μG)
10—Penicillin-Streptomycin (5,000 U/mL) (ThermoFisher scientific Cat #15070063)
11—PBS−/− (ThermoFisher scientific Cat #10010023)
12—Dimethyl Sulfoxide (Sigma-Aldrich Cat #D8418-100ML)
13—6 well cell culture plates (ThermoFisher scientific Cat #140675)

Growth medium: can be prepared and stored in 4° C. Don't keep longer than a month (date bottle).

Growth medium composition for 100 ml-Glasgow's MEM (GMEM) 92 ml. 10 ml fetal bovine serum ES cell qualified, 1 ml of Nonessential amino acid, 1 ml of sodium pyruvate, 1 ml of Penicillin-Streptomycin, 100 μl of 2-mercaptoethanol, 100 μl LIF (final is 1000 unit/ml).

### 2× Freezing Buffer—

20% Dimethyl Sulfoxide, 80% fetal bovine serum ES cell qualified was prepared and stored in −20° C. freezer.

### ESC Maintenance:

1) Add 2 ml/well of 0.1% gelatin solution and incubate at room temperature for overnight before seeding the cells.
2) Thaw TG2A cells by taking out 1 ml vial of the frozen cells (frozen in 1:1 freezing buffer and growth media) from liquid nitrogen and immediately thaw in 37° C. water bath
3) Wash the cells by adding 2 ml of growth media to the thawed vial and centrifuge the cells at 1000 RPM for 5 minutes.
4) Aspirate the media and dilute pelleted cells in 2 ml of growth medium-count cells.
5) Aspirate the gelatin from plate and seed ˜500,000 cells in 2 ml of growth medium per well of a 6 well plate.
6) Perform a complete media change every 24 hours.
7) Once cells reach ˜80% confluency-passage cells into new plates.
   a) For passaging of cells, aspirate media and rinse cells with 1 ml of PBS per well.
   b) Aspirate PBS.
   c) Add 300 μl of TrypLE express enzyme per well and incubate for four minutes in 37° C., CO₂ incubator.
   d) Take cells out of incubator and quench TrypLE express enzyme by adding 2 mL of growth medium.
   e) Centrifuge cells at 1000 RPM for 5 minutes. Aspirate the liquid and dilute cells in 2 ml of growth medium.
   f) Transfer ˜500,000 cells per well in gelatin coated 6 well cell culture plate in 2 ml of growth media.
   g) Perform a complete media change every 24 hours.
   h) After reaching the confluency about 80% repeat the same process of point 7 again.
      Differentiation of TG2A Cells into Anterior Intermediate Mesoderm Like Cells

### Reagents/Materials

1—Neurobasal Medium (ThermoFisher scientific Cat #21103049)
2—DMEM-F12 Medium (ThermoFisher scientific Cat #)
3—2-Mercaptoethanol (ThermoFisher scientific Cat #21985023)
4—Sodium pyruvate solution (Sigma Aldrich Cat #S8636-100ML)
5—MEM Non-Essential Amino Acids (ThermoFisher scientific Cat #11140050)
6—N-2 Supplement (ThermoFisher scientific Cat #17502048)
7—B-27 Supplement (ThermoFisher scientific Cat #17504044)
8—Glutamax (100×) (ThermoFisher scientific Cat #35050061)
9—Knockout serum replacer (ThermoFisher scientific Cat #10828010)
10—Penicillin-Streptomycin (5,000 U/mL) (ThermoFisher scientific Cat #15070063)
11—TrypLE Express Enzyme (ThermoFisher scientific Cat #12604013)
12—ActivinA (R&D systems Cat #338-AC-010)
13—bFGF (Proteintech Cat #HZ-1285)
14—Retinoic Acid (Sigma-Aldrich Cat ##R2625)
15—6 well cell culture plates (ThermoFisher scientific Cat #140675)
16—ROCK inhibitor Y-27632 (Enzo Life Sciences Cat #ALX-270-333)
17—PBS−/− (ThermoFisher scientific Cat #10010023)

Priming medium: can be prepared and stored in 4° C. Don't keep longer than a month (date bottle).

Priming medium composition for 100 ml medium: add 46.9 ml of Neurobasal Medium, 46.9 ml of DMEM-F12 Medium, 500 μl of N2 supplement. 1 ml B27 supplement, 500 μl of Glutamax, 100 μl 2-Mercaptoethanol, 1 ml Sodium pyruvate, 1 ml knockout serum replacer, 1 ml of non-essential amino acids, 1 ml of Penicillin-Streptomycin. The additional growth factors, 10 ng/ml Activin A and 10 ng/ml bFGF are mixed in medium just before the addition of medium into the cells.

### AIM Differentiation Medium

A small amount of basal AIM differentiation media can be prepared and stored in 4° C. Don't keep longer than a month (date bottle).

AIM differentiation medium composition for 100 ml of medium: add 92.9 ml of DMEM-F12 medium, 1 ml of Sodium pyruvate, 4 ml of knockout serum replacer, 1 ml of non-essential amino acids, 100 μl of 2-Mercaptoethanol, 1 ml of Penicillin-Streptomycin. Addition of growth factors 10 ng/ml Activin A and 100 nM RA are mixed in medium right before the addition of medium into the cells.

### 1) Expanding Cells for a Differentiation

The starting cells need to be in undifferentiated and in the growth phase. To make starting cells are in same growth phase consistently between experiments, Harvest cells from 80% confluent plate (at least one passage after thawing).
a—Aspirate growth media and wash cells with 1 ml of PBS−/− of 6 well plate.
b—Aspirate PBS−/− and incubate cells with 300 μl of TrypLE express per well of 6 well plate for 5 minutes in CO₂ incubator.
c—Take out cells from incubator and quench TrypLE express enzyme by adding 2 mL of growth medium.
d—Centrifuge the cells at 1000 RPM for 5 minutes, aspirate the liquid and dilute the pelleted cells in growth medium.
e—Seed ˜300,000 cells per well of gelatin coated 6 well plate in 2 ml of growth medium.
f—Change the complete media with fresh growth media in every 24 hours
g—After 48 hours of the seeding, the cells are ready to begin differentiation.

### 2) Start Differentiation

a—Aspirate the growth medium from 48 hours grown cells and wash with 1 ml of PBS−/− per well of six well plate.
b—Aspirate PBS−/− and incubating cells with 300 μl of TrypLE express per well of 6 well plate for 5 minutes in CO₂ incubator.
c—Take out cells from incubator and quench TrypLE express enzyme by adding 2 mL of growth medium.
d—Centrifuge the cells at 1000 RPM for 5 minutes, aspirate the liquid and dilute the palleted cells in priming media.
e—Seed ˜30,000 cells per well of gelatin coated 6 well plate in 2 ml of priming medium and incubate the cells in CO₂ incubator.
f—Change complete priming media after 24 hours of incubation. Since cells loosely attached to the surface the medium was exchanged very carefully without disturbing the plate much.
g—After 48 hours of total incubation in priming medium, change complete media with 2 ml of AIM differentiation media per well of 6 well plate. Since cells loosely attached to the surface, the medium is exchanged very carefully without disturbing the plate much.
h—At 72 hours of total incubation, change complete media with 3 ml of AIM differentiation media per well of 6 well plate with 5 μM Y-27632.
i—At 96 hours of total incubation cells are ready for dissociation and are ready for organoid generation.

Note: cells were collected every day after second day of differentiation for RNA isolation. qPCR was done for multiple AIM markers, gonadal markers, pluripotency marker Oct4, posterior intermediate mesoderm marker HoxD11. The data was confirmed by immunostaining of AIM as well as gonadal markers Wt1, gonadal marker Sox9.

### Generation of Testicular Organoids Using 4 Day Differentiated AIM Cells

### Materials

1—DMEM-F12 Medium (ThermoFisher scientific Cat #)
2—2-Mercaptoethanol (ThermoFisher scientific Cat #21985023)
3—Sodium pyruvate solution (Sigma Aldrich Cat #S8636-100ML)
4—MEM Non-Essential Amino Acids (ThermoFisher scientific Cat #11140050)
5—Glutamax (ThermoFisher scientific Cat #35050061)
6—Knockout serum replacer (ThermoFisher scientific Cat #10828010)
7—Penicillin-Streptomycin (5,000 U/mL) (ThermoFisher scientific Cat #15070063)
8—FGF9 (R&D systems Cat #273-F9-025)
9—Retinoic Acid (Sigma-Aldrich Cat ##R2625)
10—IGF1 (Sigma-Aldrich cat #13769)
11—Insulin (Sigma-Aldrich cat #19278)
12—Prostaglandin D2 (Cayman Chemical Cat #12010)
13—ROCK inhibitor Y-27632 (Enzo Life Sciences Cat #ALX-270-333)
14—Matrigel basement membrane matrix (Corning 354234)
15—TrypLE Express Enzyme (ThermoFisher scientific Cat #12604013)
16—Aggrewell 400, 24 well plate (Stemcell Technologies Cat #34421)
17—Anti-Adherence Rinsing Solution (Stemcell Technologies Cat #07010)
18—BMP4 (R&D 314-BP)
19—IWR1 (Sigma-Aldrich Cat #681669)
20—EGF (Sigma-Aldrich Cat #E4127)

Organoid differentiation medium: A small amount of basal organoid differentiation media can be prepared and stored in 4° C. Don't keep longer than a month (date bottle).

Combination 1: Organoid differentiation medium composition for 100 ml of medium: add 92.9 ml of DMEM-F12 medium, 1 ml of Sodium pyruvate, 4 ml of knockout serum replacer, 1 ml of non-essential amino acids, 100 μl of 2-Mercaptoethanol, 1 ml of Penicillin-Streptomycin. Additional factors, 1% Matrigel, 100 nM Retinoic Acid, 17 nM IGF1, 100 nM Insulin, 200 ng/ml FGF9, 500 ng/ml PGD2, 5 μM Y-27632 was mixed right before adding the media in the cells.

Combination 2: Organoid differentiation medium composition for 100 ml of medium: add 92.9 ml of DMEM-F12 medium, 1 ml of Sodium pyruvate, 4 ml of knockout serum replacer, 1 ml of non-essential amino acids, 100 μl of 2-Mercaptoethanol, 1 ml of Penicillin-Streptomycin. Additional factors, 1% Matrigel, 100 nM Retinoic Acid, 17 nM IGF1, 100 nM Insulin, 1 ng/ml FGF9, 500 ng/ml PGD2, 20 ng/ml BMP4, 50 ng/ml EGF, 5 PM Y-27632 was mixed right before adding the media in the cells.

Combination 3: Organoid differentiation medium composition for 100 ml of medium: add 92.9 ml of DMEM-F12 medium, 1 ml of Sodium pyruvate, 4 ml of knockout serum replacer, 1 ml of non-essential amino acids, 100 μl of 2-Mercaptoethanol, 1 ml of Penicillin-Streptomycin. Additional factors, 1% Matrigel, 100 nM Retinoic Acid, 17 nM IGF1, 100 nM Insulin, 1 ng/ml FGF9, 500 ng/ml PGD2, 20 ng/ml BMP4, 50 ng/ml EGF, 2 μM IWR1, 5 M Y-27632 was mixed right before adding the media in the cells.

### Preparing Plates for Organoid Differentiation

Before starting the organoid differentiation, the aggrewell 400-24 well plate was prepared as follow.
a—Add 500 μl of anti-adherence rinsing solution per well of aggrewell 400-24 well plate and incubate for 30 minutes at room temperature.
b—Aspirate anti-adherence rinsing solution and wash well with 1 ml of PBS−/− per well.
c—Aspirate PBS−/− and add 500 μl of organoid differentiation medium per well of aggrewell 400-24 well plate.
d—Centrifuge the plate for 5 minutes at 500 RCF to remove the trapped air in the well. Now plates are ready for seeding of the cells.

### 1—Organoid Differentiation

a—Aspirate the AIM differentiation media and wash the cells 3 times with 2 ml of PBS−/− per well of six well plate from 4 day differentiated AIM cells.
b—Add 500 μl of TrypLE Express Enzyme per well of six well plate and incubate for 5 minutes in CO₂ incubator.
c—Take out cells from incubator and quench TrypLE express enzyme by adding 2 mL of growth medium per well of six well plate.
d—Centrifuge the cells at 1000 RPM for 5 minutes, aspirate the liquid and dilute the palleted cells in organoid differentiation medium.
e—About 300,000 cells was mixed in 500 μl of organoid differentiation media and added to preprepared aggrewell plate per well.
f—Incubated plate for 10 minutes at CO₂ incubator to let cells settle down in the wells.
g—Centrifuge aggrewell plate again for 5 minutes at 500 RCF.
h—Incubate cells in CO₂ incubator.
i—The forming organoids was undisturbed for 2 days and then after half of medium was exchanged with fresh organoid differentiation media every day.
j—The organoids were collected on total 6 days and 8 days of differentiation from day 0 for RNA isolation and qPCR analysis different terminally differentiation markers.
k—Data was confirmed by whole mount immunostaining of progenitor marker CoupTf2, Sertoli cell markers Gata4, Sox9 and Wt1, Leydig cell marker Cyp17a1.

### Results

1. Generation and validation of mouse testis-like organoids from mESC. We have leveraged genetic and scRNAseq data from mice to develop and optimize a mouse ESC differentiation protocol to produce cells resembling many of the testicular cell types. Our approach entails iteratively titrating cells' seeding density and testing many combinations of growth factors, cytokines, and small molecules at multiple concentrations and durations, until we produced cells that recapitulate those along the differentiation trajectory observed in vivo (FIG. 1). During this protocol optimization efforts we collected cells at various time-points and monitored the expression of multiple markers described in FIG. 1 to determine if our in vitro derived cells traverse through expected stages of differentiation: epiblast state (OCT4, FGF5, FGF8 and DNMT3b), presomatic mesoderm (T and TBX6), anterior intermediate mesoderm (LHX1, GATA3), gonadal primordium (WT1, GATA4, EMX2, LHX9), and terminally differentiated testis somatic cells such as the Sertoli (Sox9⁺, Gata4⁺, SF1⁺, WT1⁺ and AMHR2⁺), Leydig (SF1, 3BHSD, STAR), and Myoid (SMA, TAGLN) cells. Applying similar strategies for mouse and human ESCs we have established two slightly different cocktails.

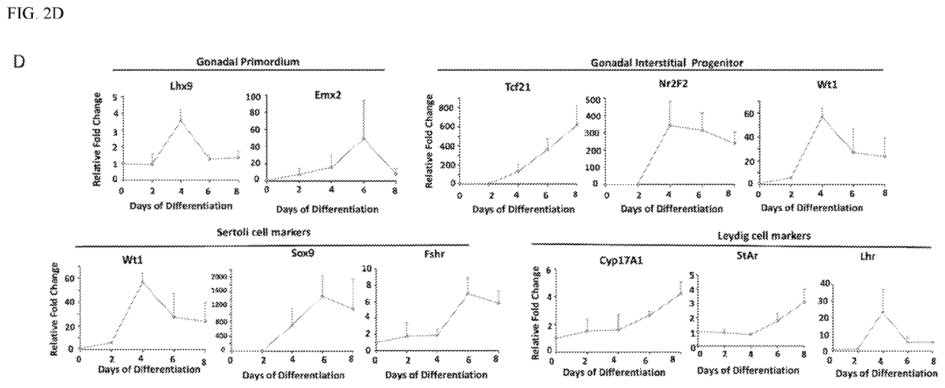

Briefly, treating mouse ESCs with Activin A and bFGF for two days leads to a suppression of many pluripotency marker (Nanog, Sox2, Rex1, Klf1, FIG. 2B left), while maintaining Oct4 expression as well as gaining expression of Fgf5, Fgf8 and Bracury (T) (FIG. 2B right). These transcriptional changes are consistent with the ESCs transitioning to epiblast cells (FIG. 1). Once in the epiblast state, cells were treated with Activin A and Retinoic Acid for 3-5 days (FIG. 2A). During the 3-5 day differentiation window, early mesoderm markers such as OSR1 decrease while anterior intermediate mesoderm and gonadal primordium markers are enriched—e.g. Gata4, Pax2, WT1, Sox9 and Lhx1 (FIG. 2C). Importantly, our differentiation protocol restricts the generation of posterior intermediate mesoderm progenitors (HOXD11+ cells), which give rise to the kidney—a bifurcating differentiation decision (FIG. 2C). Although these preliminary findings at day 5 were exciting, the cells in monolayer rarely formed organoids spontaneously. Therefore, to promote organoid formation and further push toward advanced gonadal cell fate beyond the gonadal primordium, we dissociated the cells at either 4 or 5 days and replated ˜300K cells in 800 microwell Aggrewells (FIG. 2A). The plated cells were supplemented with Fgf9, Igf1, Insulin, Pgd2, and RA (FIG. 2A). Within 24 hrs after plating, the single cell suspensions formed spheroids/organoids, which were maintained for an additional 48-96 hours in culture. RT-qPCR analysis of 6 to 8-day organoids reveals an increase in interstitial progenitor marker (Tcf21, Nr2f2, Wt1) and Sertoli cell markers (Wt1, Sox9, Fshr) as well as the identification of Leydig cell markers (Cyp17a1, StAr and LHR) (FIG. 2D).

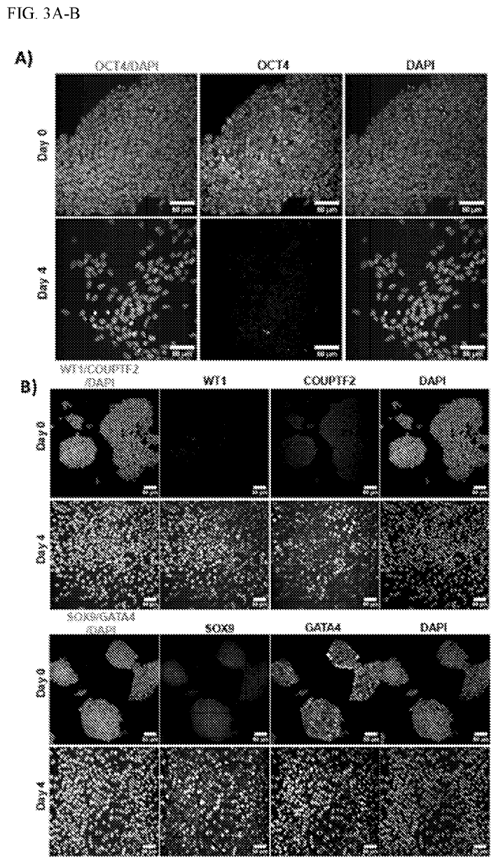

To confirm that the changes in RNA are evident on protein levels, we performed immunofluorescence imaging using a subset of markers analyzed by qPCR. As expected, OCT4, a marker for stemness, is expressed in day 0 but repressed in day 4 (FIG. 3A). Meanwhile, cells at Day 4 begin expressing gonadal primordium markers like Wt1, CoupTF2 (Nr2F2), Sox9) and maintain expression of Gata4 (Gata4 is expressed in mouse ESCs but not human) (FIG. 3B). After dissociation and aggregation, Retinoic acid was required for an efficient differentiation and complete suppression of the pluripotency program (data not shown), as well as the generation of an ordered tubule-like structure within the organoid (FIG. 3C). This tubular organization was noted in roughly 70% of the organoids generated in more than 10 replicate experiments. Within the tubule organization centers multiple Sertoli cell like markers such as Gata4/Sox9/AMH/SF1 are detected, and furthermore like in vivo cells these cells secrete extracellular matrix (Lamin) and form tight functions (Zo1: FIG. 3C).

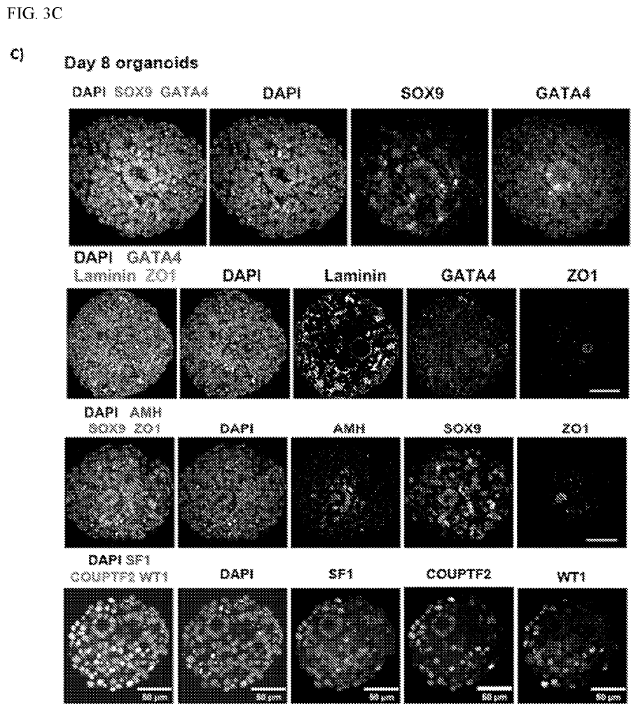

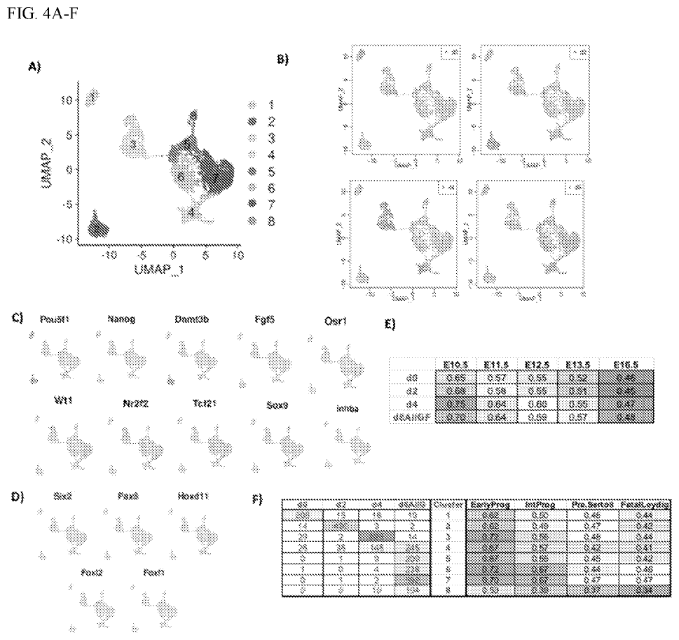

2. The mouse in vitro derived testis somatic-like cells resemble gonadal progenitors identified in E10.5-11.5 gonads. For mouse ESC experiments, cells from days 0), 2, 4, and 8 of differentiation were molecularly barcoded, captured, and sequenced in the same run to avoid batch effect. Unsupervised clustering of ˜5.000 cells revealed 8 clusters: C1-C8 (FIG. 4A). Genes highly expressed in C1 cells include pluripotency factors such as Oct4, Sox2, and Nanog (FIG. 4C), consistent with this cluster being ESCs. C2 maintains high Oct4 levels, expresses DNMT3b, Fgf5/8, but is depleted of Nanog, consistent with Epiblast cells. C3-8 cells are molecularly heterogenous and enrich for anterior intermediate mesoderm and gonadal primordium cell populations (FIG. 4C). Importantly. C1 cells are mainly derived in day 0, while Clusters 2, 3, and 4-8 are derived days 2, 4, and 8 respectively (FIG. 4B, 4F). Comparisons of our in vitro derived somatic cells with those reported for in vivo cells (12, 13) reveal a parallel advance of our day 0-2-4-8 cells and the E10.5-E13.5 embryonic gonadal cells (FIG. 4E), and our eight clusters of cells, advancing in 0-8 days, matches the in vivo data's gonadal progenitors, interstitial progenitors, and pre-Sertoli cells (FIG. 4F). Importantly, our differentiation protocol appears specific for gonadal cells, as transcription factors for the alternative urogenital cell fates are not detected, e.g., the kidney (Hoxd11, Six2, nphs2), adrenal (Sult2a1; Arhgap), ovary (Foxf1), or granulosa cells (Fox12) (FIG. 4D).

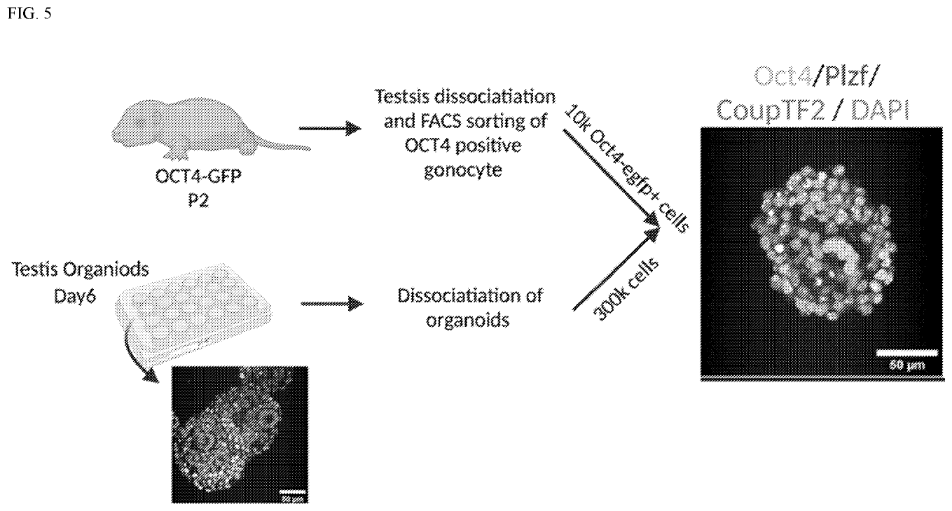

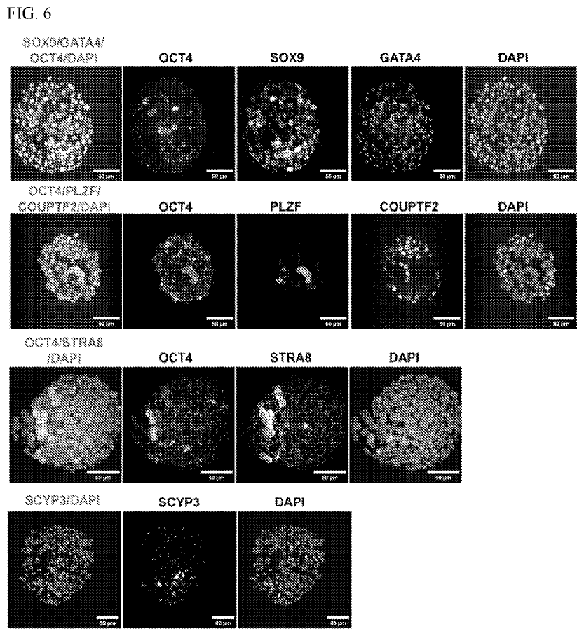

3. The mouse testis somatic like cells can support the maintenance of pro-spermatogonia in vitro and possibly their differentiation as well. To determine whether the in vitro derived somatic like cells can support germ cell maintenance and differentiation, we performed a similar differentiation protocol described above except we collected and dissociated organoids at day 6 and re-aggregated the in vitro derived somatic cells with roughly 4% pro-spermatogonia (Oct4-egfp⁺ cells) from the postnatal days (PND) 1-2 testis (FIG. 5). The combined somatic and germ cell organoids were maintained in culture for an additional 2, 4, and 6 days (a total of 10-16 days from Day-0 ESCs). Importantly, these in vitro assembled organoids can incorporate pro-spermatogonia within these structures, sustain pro-spermatogonia germ cells (Oct4-EGFP+/Plzf) (FIG. 6), but also possibly accommodate pro-spermatogonia differentiation as seen by the appearance of Oct4-Egfp-dim cells and Stra8 and Sycp3 cells in later days in culture. With this initial success, our ongoing experiments continue to finetune meiotic progression and completion.

Based on our current data, we have very strong molecular and histological evidence suggesting that our testis-like organoids resemble fetal like gonadal tissue. Importantly these cells support spermatogonia and primordial germ cell like cell homing, proliferation, and differentiation, and we are currently optimizing meiotic progression.

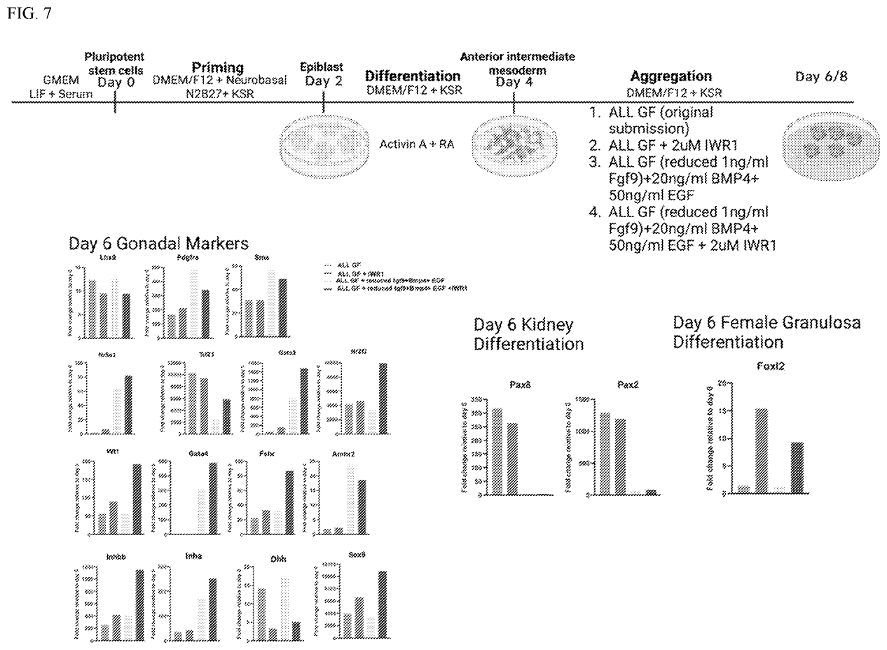

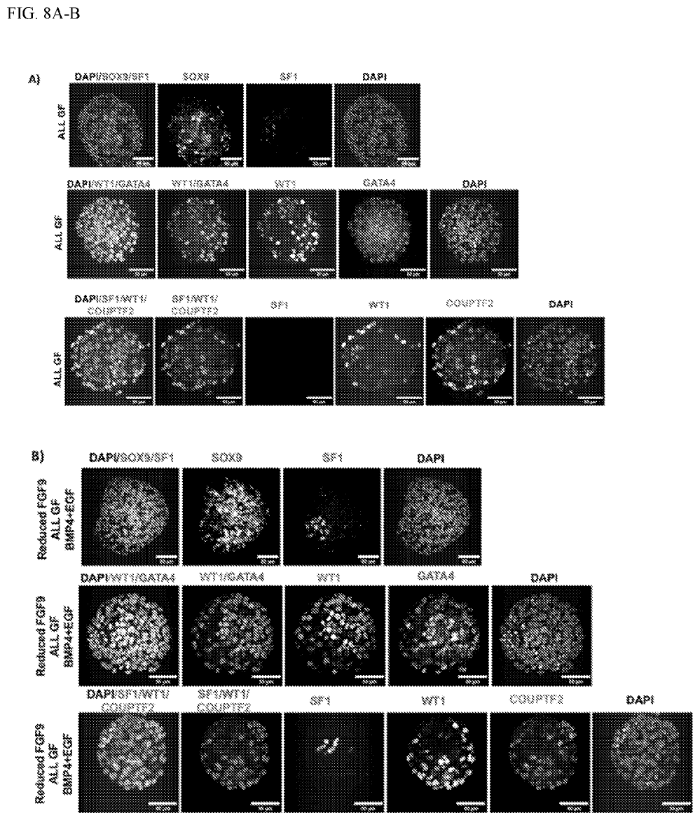

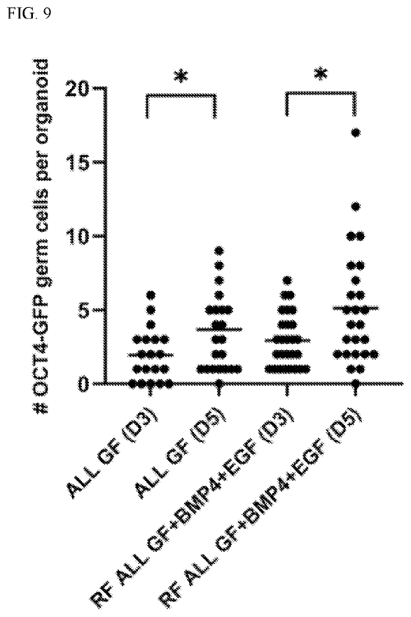

4. Efforts to continue to refine the robustness of our somatic cell differentiation protocol. Although our original cocktail in FIG. 2A worked well and consistently, we often detect off-target differentiated cells (like kidney cells) in our organoids at low frequency. To enhance the purity and suppress any off-target differentiation, we screened an additional 20 drugs in our ALL-GF baseline media. Adding BMP4+EGF1 to our ALL-GF original cocktail while reducing FGF9 concentration (referred to as cocktail 3) was able to significantly improve testis-like differentiation while completely blocking off-target changes (FIG. 7A). Specifically, all gonadal markers (Lhx9, Pdgra, Sma, Nr5a1, Tcf21, Gata3, Nr2f2, Wt1, Gata4, Fshr, Amhr, Inhbb, Inha, Dhh and Sox9) are elevated in cocktail 3 in comparison to ALL GF. Importantly, this cocktail suppresses kidney and granulosa cell (female lineage) gene expression. Consistent with the improved RNA expression, many of the protein markers were more robustly expressed by immunofluorescence as well (FIG. 8). Furthermore, these cells are better at expanding germline stem cell progenitors in vitro (FIG. 9).

### Example 2

The following example provides a description of the reagents and protocols for producing artificial Sertoli cells according to the present invention from human Embryonic Stem Cells (hESC). The protocol can be preferably be continued to at least 13 days and up to 22 days.

### Reagents.

STEMdiff APEL Medium (Stem Cell Technologies, Cat. No: 05270 or 05275).
DMEM/F-12 (Thermo Fisher Scientific, Cat. No: 11320-082).
DMSO (Sigma Aldrich, Cat. No: D5879).
Dulbecco's phosphate-buffered saline (DPBS) (Thermo Fisher Scientific, Cat. No: 14190-144).
Accutase (Cat No: 07920)
CHIR99021 (R&D, Cat No: 9902): Stock Solution (10 mM)—Centrifuge the tube briefly before opening. Reconstitute 10 mg of CHIR99021 into 2.149 ml of DMSO to make 10 mM stock. Aliquot it into 20 ul and label “Ch” store them at −20° C. Do not reuse after 24 hr after thawing.
Y27632 ROCK inhibitor (Enzo, Cat No: ALX-270-333): Stock Solution (5 mM)—Dissolve 1 mg in 625 uL sterile TC water. Aliquot into 20 uL. Label “Y”. Store at −20° C. at stem cell core. Dilute in 10 mL culture medium for final 10 μM. Use immediately after thawing. Do not reuse after 24 hr after thawing.
Heparin (Sigma Aldrich, Cat. No: H4784-250 Mg): Stock solution (1 mg ml⁻¹)—Reconstitute to 1 mg ml⁻¹ in ultrapure water and filter sterilize it through a polyethersulfone (PES) 0.22 μm syringe-driven filter unit. Heparin solution can be stored at 4° C. for more than 12 months. Aliquots in 40 μl. Store at 4° C.
FGF9 (R&D, Cat No: 273-F9-025): Stock Solution FGF9 (100 μg ml⁻¹)—Centrifuge the tube briefly before opening. Reconstitute to 100 μg/ml in filtered DPBS containing 0.1% (wt/vol) human serum albumin. Aliquot it into 20 μl and store them at −80° C. for up to 6 months. Store stock at −80° C. FGF9 can be stored at 4° C. for up to 2 weeks once it is thawed.
RA (Sigma, Cat No: R2625-5 mg): Stock Solution (0.5 mM)—1 mg of RA was weighted and dissolve in 3 ml of the DMSO, which makes the concentration of 1.11 mM Stock Solution. Serial dilute it to 0.5 mM Stock and store at −20° C. in 50 μl or 100 μl Aliquots. Use 0.5 mM of stock solution to the cell culture media based on appropriate concentration desired for experiment. Do not reuse after 24 hr after thawing.
Insulin (Sigma, Cat No: 19278): Stock Solution (1.7 mM) in liquid form from vendor.
IGF1-50 μg (Sigma, Cat No: 13769): Stock solution (10 mM)—50 ug of IGF1 is dissolved in 658 μl of 0.2% acetic acid which makes 10 mM stock conc. Aliquots into 10 μl and store at −80 C. Take 10 μl of 10 mM and dissolve in 1 ml of 0.2% acetic acid which make 100 μM concentration to the cell culture media based on appropriate concentration desired for experiment. After thawing it is good for 1 month at 4° C.
PGD₂ (Cayma, Cat No: 12010): Stock Solution (2 mg/ml)—Dissolve PGD₂ in 200 μl pbs 7.4 makes 2 mg/ml of stock solution and aliquoted in 5 ul and stored at −80° C. Do not reuse after 24 hr after thawing.
Matrigel (Corning, Cat No: 354234, Lot no: 9133008): Stock Solution (2 mg/ml or 1 mg/ml)—Dilute the Matrigel to desired concentration 2 mg/ml or 1 mg/ml in DMEM/F12 media and aliquot them and store at −20° C. until use.
Epidermal Growth Factor from murine submaxillary gland, EGF (Sigma, cat no. E4127-. 1 mg): Stock Solution (100 μg/ml)—0.1 mg of EGF was dissolved in 10 ml of 0.1% BSA/PBS in 10 ml then aliquoted in 1 ml and store at −20° C. until use.
IWR1 (Sigma: cat no. 681669-10 mg)—Stock Solution (10 mg/ml)—Dissolve 10 mg/ml in DMSO and prepare aliquots and store at −20° C. until use.
BMP4 (R&d: cat no. 314-bp-500): Stock Solution (200 μg/ml)—Dissolve 200 μg/mL in sterile 4 mM HCl containing 0.1% BSA/H2O and prepare aliquots and store at −20° C. until use.

### Materials.

Nunc™ 4-Well Dishes for IVF (Fisher, Cat No. 144444).
Nunc Multidish Nunclon Delta SI-24 well (Fisher, Cat No: 142475) Coverslips 12 mm (Hampton Research, Cat no. HR3-277)
Bench top centrifuge (Eppendrof, 5417c)
Biological safety cabinet
CO₂ Incubator (Heraeus)
Conical tubes 15 ml (Falcon, Cat No. 352096) Conical tubes 50 ml (Falcon, Cat No. 352070)
Freezing container (Nalgene, Mr. Frosty)
Inverted constrating tissue culture microscope (KL1500CD, Lieca) Laser confocal microscope (Nikon Eclipse Ti2)
Pipettes (Gilson)
Automated cell counter (Biorad, TC20)
Serological pipettes (Fisher, Cat No, 13-678-11D, 13-676-10J, 13-676-10R)
Stericup 0.22 μm Filter unit (Millipore, 03290)
Steri filter pipette tips (Genesie Scientific, 24-815, 24-804,24-830, 26-401)
Sterile Microcentrifuge tubes (Fisher, Cat No. 05-408-120)

### Coverslip Cleaning Method:

Shake coverslips in xylene in 50 ml conical tube for 2-3 hours.
Discard xylene and rinse with acetone. Discard acetone and then shake in fresh acetone in 50 mL conical tube for 2-3 hours.
Discard acetone. Autoclave in water if desired on liquid cycle. Rinse coverslips with 100% ethanol 2 times. Store coverslips in fresh 100% ethanol at 4° C.

### Day −1 Plating ESC in Single Cell Suspension for Differentiation

### Coating Plates for Cell Pellet (RNA Seq Analysis) or Staining of Coverslip.

Transfer the coverslip with forceps to 24 or 4 well plate after cleaning and dry out until alcohol evaporates completely in a Biosafety cabinet hood under sterile conditions.
Coat coverslip with Matrigel in plate for 1 hour before plating the cells.
Coat the 24 or 4 well plate with Matrigel for 1 hour before plating the cells

### Single Cell Suspension for Monolayer

60 mm petri plates are cleaned to remove any differentiating colonies.
Wash with 2 ml DPBS buffer.
Add 0.5 ml of accutase and place in incubator for 5-10 mins.
Add 2-5 ml of DMEM/F12 media to stop the dissociation.
Pellet cells down by centrifuging for 3 min at 1000 rpm.
Resuspend with 5 ml of mTESR media with 10 μM Rock inhibitor and count the cells.
Plate 10,000 cells per well of 24 well Matrigel coated plate.

### Day 0 ESC Cell Collection as a Control:

hESC cells are collected on day 0.
Cells are washed with 0.5 ml of DPBS buffer for each well of 24 well plate.
0.5 ml of accutase is added and the plates placed in incubator for 3-5 mins.
Add 1 ml of DMEM/F12 media to stop the digestion reaction.
Centrifuge at 10,000 g for 2 min.
Aspirate the media.
Snap freeze in Liquid N₂ and store at −80 C until later use.
Fix the cells in 4% PFA for 20 min and wash 3 times with DPBS, store in DPBS in 4° C. until use.

### Cells Used to Start Differentiation

Remove mTESR media with Rock inhibitor and add 0.5 ml of APEL2 media+CHIR 3 μM.
Media changed every 2 days.

### Day 2

Change media with APEL2 media+CHIR 3 μM.

### Day 4: Presomitic Mesoderm.

Change media with 200 ng/ml FGF9+1 μg/ml Heparin.
Change the media every two days

### Day 6

Change media with 200 ng/ml FGF9+1 μg/ml Heparin

### On Day 7 (Intermediate Mesoderm)

Some wells are collected on day 7 for analysis.
Cells are washed with 0.5 ml of DPBS buffer for each well of 24 well plate.
0.5 ml of accutase is added and the plates placed in incubator for 3-5 mins.
Add 1 ml of DMEM/F12 media to stop reaction.
Centrifuge at 10,000 g for 2 min.
Aspirate the media
Snap freeze in Liquid N₂ and store at −80 C until later use (Intermediate Mesoderm).
Fix the cells in 4% PFA for 20 min and wash 3 times with DPBS, store in DPBS in 4° C. until use.
The remaining wells are continued in the differentiation protocol: for these wells we will remove growth factors and change the media to APEL Only.

### Day 9

Change the APEL Only media.

### Day 10 (Genital Ridge)

Some wells are collected at day 10 for analysis.
Cells are washed with 0.5 ml of DPBS buffer for each well of 24 well plate.
0.5 ml of accutase is added and the plates placed in incubator for 3-5 mins.
Add 1 ml of DMEM/F12 media to stop reaction.
Centrifuge at 10,000 g for 2 min.
Aspirate the media.
Snap freeze in Liquid N2 and store at −80 C until later use.
Fix the cells in 4% PFA for 20 min and wash 3 times with DPBS, store in DPBS in 4° C. until use.
Remaining wells receive various combinations of growth factors (media will be changed every 2 days):
   Combo: APEL+17 nM IGF1+100 nM Insulin
   Combo: APEL+17 nM IGF1+100 nM Insulin+200 ng/ml FGF9.
   Combo: APEL+0.1 μM RA+100 nM Insulin+17 nM IGF1+500 ng/ml PGD₂+200 ng/ml FGF9+100 ng/ml FSH+20 ng/ml LH+1 μM Testosterone.
   Combo: APEL+0.1 μM RA+100 nM Insulin+17 nM IGF1+500 ng/ml PGD₂+1 ng/ml FGF9+10 ng/ml BMP4+50 ng/ml EGF+100 ng/ml FSH+20 ng/ml LH+1 μM Testosterone.
   Combo: APEL+0.1 μM RA+100 nM Insulin+17 nM IGF1+500 ng/ml PGD₂+1 ng/ml FGF9+10 ng/ml BMP4+50 ng/ml EGF+2 μM IWR1+100 ng/ml FSH+20 ng/ml LH+1 μM Testosterone.

### Day 12

Change the media for every two days with growth factors:
   Combo: APEL+17 nM IGF1+100 nM Insulin
   Combo: APEL+17 nM IGF1+100 nM Insulin+200 ng/ml FGF9.
   Combo: APEL+0.1 μM RA+100 nM Insulin+17 nM IGF1+500 ng/ml PGD₂+200 ng/ml FGF9+100 ng/ml FSH+20 ng/ml LH+1 μM Testosterone.
   Combo: APEL+0.1 μM RA+100 nM Insulin+17 nM+IGF1+500 ng/ml PGD₂+1 ng/ml FGF9+10 ng/ml BMP4+50 ng/ml EGF+100 ng/ml FSH+20 ng/ml LH+1 μM Testosterone.
   Combo: APEL+0.1 μM RA+100 nM Insulin+17 nM+IGF1+500 ng/ml PGD₂+1 ng/ml FGF9+10 ng/ml BMP4+50 ng/ml EGF+2 μM IWR1+100 ng/ml FSH+20 ng/ml LH+1 μM Testosterone.

### Day 14.

Some wells are collected on day 14 for analysis.
Cells are washed with 0.5 ml of DPBS buffer for each well of 24 well plate.
0.5 ml of accutase is added and the plates placed in incubator for 3-5 mins.
Add 1 ml of DMEM/F12 media to stop reaction.
Centrifuge at 10,000 g for 2 min.
Aspirate the media.
Snap freeze in Liquid N₂ and store at −80 C until later use.
Fix the cells in 4% PFA for 20 min and wash 3 times with DPBS, store in DPBS in 4° C. until use

### (Differentiated State).

Remaining wells will have a media change every two days with growth factors
   Combo: APEL+17 nM IGF1+100 nM Insulin
   Combo: APEL+17 nM IGF1+100 nM Insulin+200 ng/ml FGF9.
   Combo: APEL+0.1 μM RA+100 nM Insulin+17 nM IGF1+500 ng/ml PGD₂+200 ng/ml FGF9+100 ng/ml FSH+20 ng/ml LH+1 μM Testosterone.
   Combo: APEL+0.1 μM RA+100 nM Insulin+17 nM IGF1+500 ng/ml PGD₂+1 ng/ml FGF9+10 ng/ml BMP4+50 ng/ml EGF+100 ng/ml FSH+20 ng/ml LH+1 μM Testosterone.
   Combo: APEL+0.1 μM RA+100 nM Insulin+17 nM IGF1+500 ng/ml PGD₂+1 ng/ml FGF9+10 ng/ml BMP4+50 ng/ml EGF+2 μM IWR1+100 ng/ml FSH+20 ng/ml LH+1 μM Testosterone.

### Day 16

Change the media for every two days with growth factors:
   Combo: APEL+17 nM IGF1+100 nM Insulin
   Combo: APEL+17 nM IGF1+100 nM Insulin+200 ng/ml FGF9.
   Combo: APEL+0.1 μM RA+100 nM Insulin+17 nM IGF1+500 ng/ml PGD₂+200 ng/ml FGF9+100 ng/ml FSH+20 ng/ml LH+1 μM Testosterone.
   Combo: APEL+0.1 μM RA+100 nM Insulin+17 nM IGF1+500 ng/ml PGD₂+1 ng/ml FGF9+10 ng/ml BMP4+50 ng/ml EGF+100 ng/ml FSH+20 ng/ml LH+1 μM Testosterone.
   Combo: APEL+0.1 μM RA+100 nM Insulin+17 nM IGF1+500 ng/ml PGD₂+1 ng/ml FGF9+10 ng/ml BMP4+50 ng/ml EGF+2 μM IWR1+100 ng/ml FSH+20 ng/ml LH+1 μM Testosterone.

### Day 18 (Differentiated Cells)

Cells are collected on day 18.
Cells are washed with 0.5 ml of DPBS buffer for each well of 24 well plate.
0.5 ml of accutase is added and the plates placed in incubator for 3-5 mins.
Add 1 ml of DMEM/F12 media to stop reaction.
Centrifuged at 10,000 g for 2 min.
Aspirate the media.
Snap freeze in Liquid N₂ and store at −80 C until later use (Differentiated State).
Fix the cells in 4% PFA for 20 min and wash 3 times with DPBS, store in DPBS in 4° C. until use.

### Coverslip Staining Protocol

Fix the cells in 4% PFA for 20 min.
Wash with PBS−/− for 5 min for 3 times and store in PBS−/− at 4° C. until staining.
Quick wash with PBS−/− before staining for 3 times before staining.
Add 200 μl in 24 well plate and incubate for 1 hour in a covered humidity chamber at RT
Blocking buffer PBS+/+, 0.5% Triton-X 100, 5% Donkey serum or goat serum and 10% Na-Azide
(8.85 ml PBS+/+, 50 μl Triton-x 100+1 ml Donkey serum or goat serum+ 100 μl Na-Azide). Add 200 μl of Primary Ab in a covered humidity chamber at 4° C. for Overnight.
Dilute the blocking buffer to 50% for primary antibody buffer (PBS+/+, 0.25% Triton-x, 2.5% Donkey serum or goat serum+50 μl NA-Azide).
Next day wash the slides with PBS−/− & 0.5% Triton-X for 10 min each.
Add 200 μl of Secondary Ab along with DAPI for 1.5 hour at room temperature in a covered humidity chamber.
(PBS+/+, 1 ml Donkey serum+100 μl Na-Azide)
Wash with PBS−/− for 10 min each.
Mount on coverslip with Vecta Shield

### Results

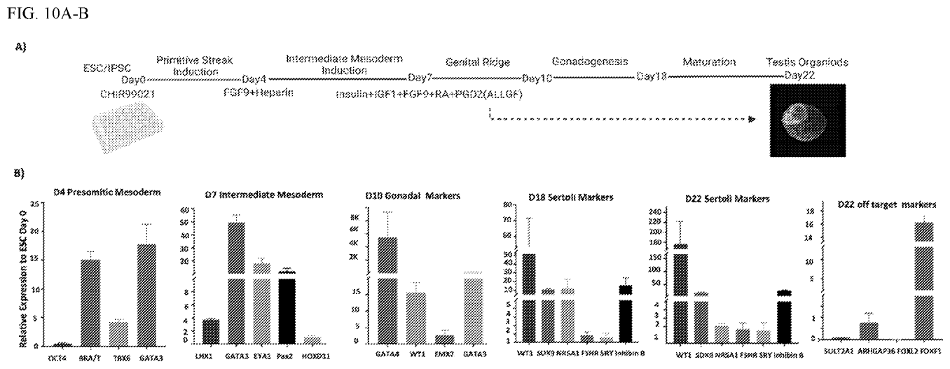

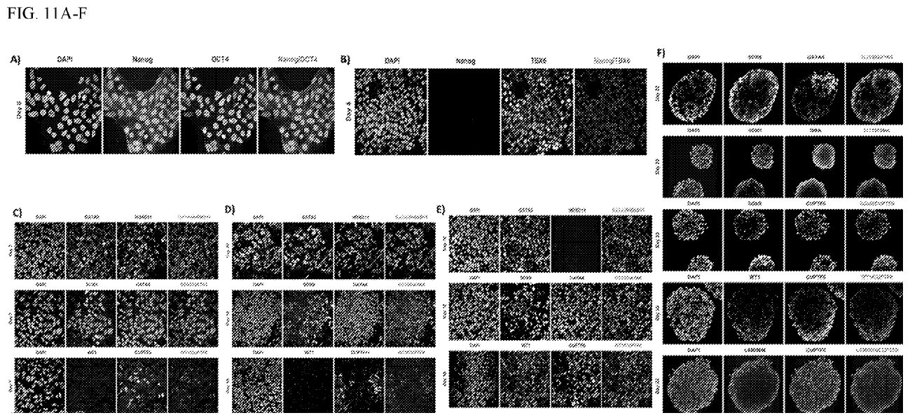

We have begun optimizing the differentiation protocol using two male human ESC lines (U6 and H1). The differentiation schema corresponding to the protocol above is described in FIG. 10A. Briefly, this protocol directs the differentiation of hESC to pre-somatic mesoderm (BRA/T, TBX6, GATA3), anterior intermediate mesoderm (LHX1, GATA3, PAX2), and the gonadal primordium cells (GAT4, WT1, EMX2), then to Sertoli (SOX9, WT1, SRY, Inhibin B, FSHR) and Interstitial cells (WT1, SF1, COUPTFII), with 40-50% differentiation efficiency. Unlike in mouse, human ESC differentiation takes longer (˜18 days); and very few organoids self-assemble spontaneously by day-18 in the 2D culture. We dissociated the cells at day 18, re-plated them in Aggrawel and were able to generate hundreds of organoids by Day 22. These organoids express multiple gonadal and Sertoli markers, but unlike the mouse ESC-derived organoids, they rarely formed organized tubules (FIG. 11). Encouragingly, the differentiation schema in humans has very little off-target expression.

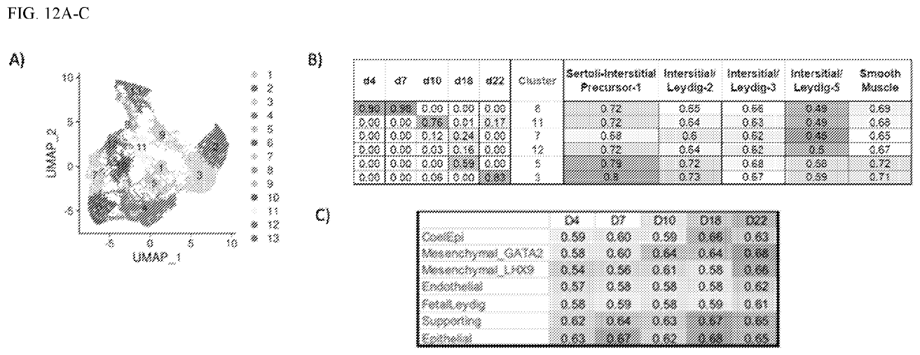

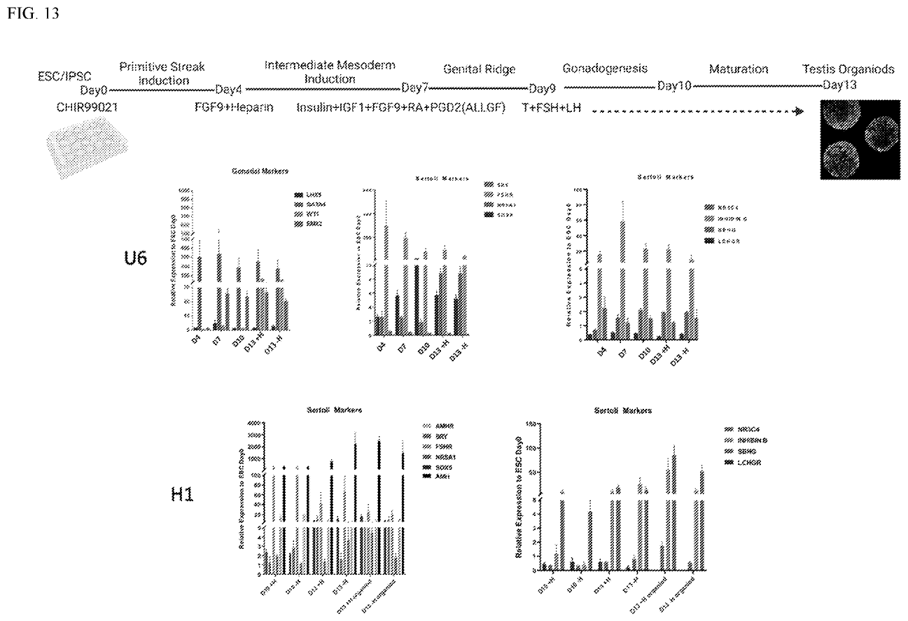

To assess the developmental stage of cultured cells we performed scRNAseq analysis across the days of differentiation. We identified 13 clusters (FIG. 12A), some of which are “early”—present on days d4 or d7, while others are emerging “late”—appearing on d10, d18, or d22 (FIG. 12B: left panel). We compared our cells, either by day or by cluster, to the in vivo scRNAseq data for embryonic gonadal cells reported recently by two other groups (14, 15). First, the early- and late-arriving cell clusters in our human ESC culture show increasing correlations with several differentiated cell types identified by the Clark group (14), such as Sertoli interstitial precursors, interstitial Leydig cell precursors, and smooth muscle subtypes (FIG. 12B: right panel). Second, when comparing our cell centroids-by-day with the cell types identified in vivo by the Roser group (15) we found that cells that emerged later in culture (e.g., Day 18 and 22) show increasing resemblance to coelomic epithelial like cells, mesenchymal interstitial cells, and supporting stromal cells (FIG. 12C). While many of the annotated cell names differ between the Clark and the Roser datasets, the comparative analysis shown above strongly suggest that our in vitro differentiation protocol has advanced ESCs forward and generated multiple testis-like somatic cell precursor cells.

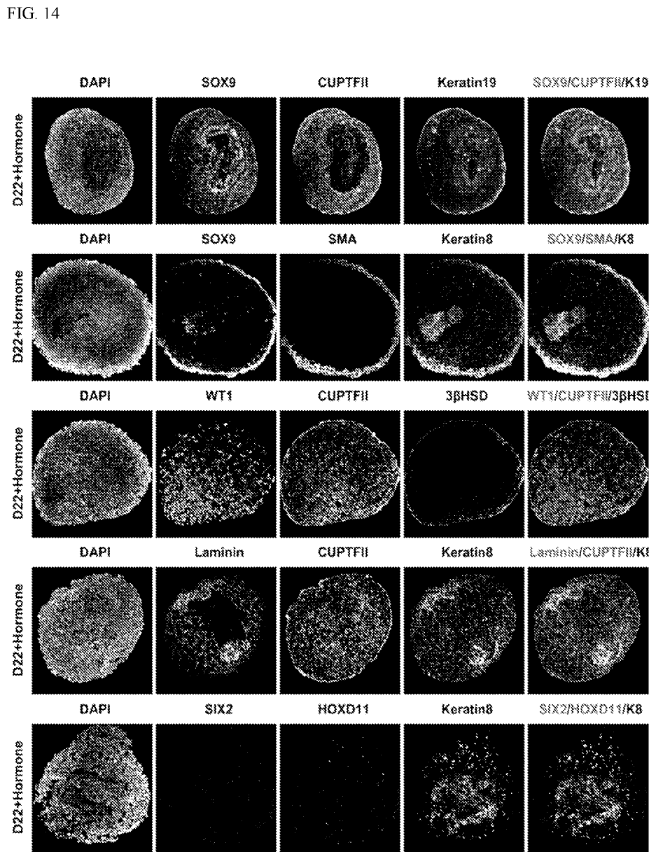

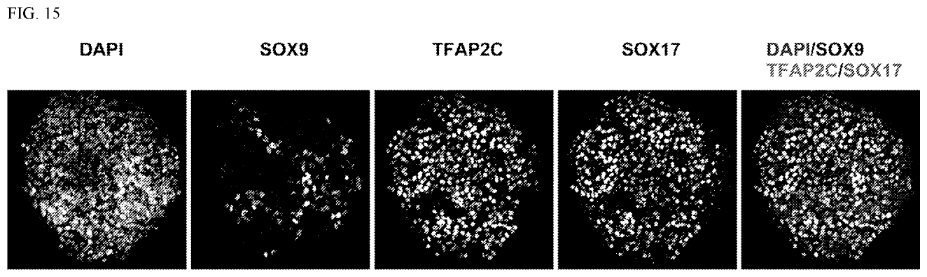

Despite this exciting progress, the human differentiation protocol generated mainly progenitor-like cells, few of which advance further to become terminally differentiated gonadal cells. Indeed, cells in our late-stage culture resembled in vivo somatic cells seen at 6-7 weeks of gestation. To drive differentiation further in human cell culture we opted to supplement the ALL-GF differentiation cocktail with hormones Testosterone. LH and FSH (FIG. 13A). Surprisingly, while the addition of hormones to mouse cells did not influence the 8-day differentiation (not shown), it significantly improves human differentiation efficiency, and the accelerated differentiation rate allowing us to detect many of the terminal differentiation markers by 13 days (FIG. 13A). RT-qPCR analysis of gonadal. Sertoli, and Leydig cell markers increase in the presence of hormone at day's 10 and 13 in both U6 and H1 embryonic stem cell (FIG. 13B). Furthermore, immunofluorescence staining in both U6 and H1 confirms a stronger induction of testis somatic cell-like markers in the organoids (FIG. 14C). Given these findings we settled on the addition of hormones for the human organoid differentiation. The analysis of day 22 organoids by immunofluorescence confirms the presence of many testis markers.

Next, we tested the ability of these in vitro derived cells to home germ cells and allow their survival. We generated PGCLC (SOX17+; TFAP2C+) and combined with our in vitro derived somatic cells at day 8, and like mouse we find that the germ cells get incorporated into the organoids and they can continue to proliferate for at least 5 days in culture (FIG. 15).

Taken together, our data demonstrate the successful generation of human somatic cells using two ESC lines. Furthermore, our in vitro derived somatic cells can home germ cells and support their proliferation in the absence of a PGC culture cocktail.

To have more clinical relevance, we next tested whether our ESC differentiation schema can be directly applied to differentiate induced pluripotent stem cells into testis somatic cells. We applied the exact regimen used for ESC, but unfortunately the efficiency was significantly lower. Unexpectedly, we realized that our initial differentiation step for IPSC required a higher concentration of Chiron (8 μM) than what is used for ESC (3 μM), and the ideal stopping point for the differentiation is 10 days. Beyond the 10 day gonadic differentiation we are decreasing the number of gonadal cells (FIG. 16).

### REFERENCES

1 Jemal, A. et al. Cancer statistics, 2004. CA Cancer J Clin 54, 8-29, doi: 10.3322/canjclin.54.1.8 (2004).
2 Gratwohl, A. et al. Hematopoietic stem cell transplantation: a global perspective. JAMA 303, 1617-1624, doi: 10.1001/jama.2010.491 (2010).
3 Wallace, W. H. Oncofertility and preservation of reproductive capacity in children and young adults. Cancer 117, 2301-2310, doi: 10.1002/cncr.26045 (2011).
4 Lambertini, M. et al. Cancer and fertility preservation: international recommendations from an expert meeting. BMC Med 14, 1, doi: 10.1186/s12916-015-0545-7 (2016).
5 Nieman, C. L. et al. Fertility preservation and adolescent cancer patients: lessons from adult survivors of childhood cancer and their parents. Cancer Treat Res 138, 201-217, doi: 10.1007/978-0-387-72293-1_15 (2007).
6 Hayashi, K., Ohta, H., Kurimoto, K., Aramaki, S. & Saitou, M. Reconstitution of the mouse germ cell specification pathway in culture by pluripotent stem cells. Cell 146, 519-532, doi: 10.1016/j.cell.2011.06.052 (2011).
7 Hayashi, K. et al. Offspring from oocytes derived from in vitro primordial germ cell-like cells in mice. Science 338, 971-975, doi: 10.1126/science.1226889 (2012).
8 Ishikura, Y. et al. In vitro reconstitution of the whole male germ-cell development from mouse pluripotent stem cells. Cell Stem Cell, doi: 10.1016/j.stem.2021.08.005 (2021).
9 Yamashiro, C. et al. Generation of human oogonia from induced pluripotent stem cells in vitro. Science 362, 356-360, doi: 10.1126/science.aat1674 (2018).
10 Sosa, E. et al. Differentiation of primate primordial germ cell-like cells following transplantation into the adult gonadal niche. Nat Commun 9, 5339. doi: 10.1038/s41467-018-07740-7 (2018).
11 Hwang, Y. S. et al. Reconstitution of prospermatogonial specification in vitro from human induced pluripotent stem cells. Nat Commun 11, 5656, doi: 10.1038/s41467-020-19350-3 (2020).
12 Stevant, I. et al. Dissecting Cell Lineage Specification and Sex Fate Determination in Gonadal Somatic Cells Using Single-Cell Transcriptomics. Cell reports 26, 3272-3283 e3273, doi: 10.1016/j.celrep.2019.02.069 (2019).
13 Stevant, I. et al. Deciphering Cell Lineage Specification during Male Sex Determination with Single-Cell RNA Sequencing. Cell reports 22, 1589-1599, doi: 10.1016/j.celrep.2018.01.043 (2018).
14 Guo, J. et al. Single-cell analysis of the developing human testis reveals somatic niche cell specification and fetal germline stem cell establishment. Cell Stem Cell 28, 764-778 e764, doi: 10.1016/j.stem.2020.12.004 (2021).
15 Luz Garcia-Alonso et al. Single-cell roadmap of human gonadal development. Nature, doi: doi: 10.1038/s41586-022-04918-4. (2022).
16 Shen, Y.-c. et al. TCF21+ mesenchymal cells contribute to testis somatic cell development, homeostasis, and regeneration in mice. Nature Communications 12, 3876, doi: 10.1038/s41467-021-24130-8 (2021).
17 Yokonishi, T., McKey, J., Ide, S. & Capel, B. Sertoli cell ablation and replacement of the spermatogonial niche in mouse. Nat Commun 11, 40, doi: 10.1038/s41467-019-13879-8 (2020).

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

1. An in vitro method for production of Sertoli cells from vertebrate pluripotent stem cells comprising:
deriving genital ridge cells from pluripotent stem cells;
treating the genital ridge cells with a base medium comprising fibroblast growth factor 9 (FGF9), insulin and/or IGF1 so that the genital ridge cells differentiate into Sertoli cells.

2. The method of claim 1, wherein the step of deriving genital ridge cells further comprises:
providing vertebrate pluripotent stem cells in a maintenance medium comprising a ROCK inhibitor;
at day 0, removing the maintenance medium comprising a ROCK inhibitor and culturing the vertebrate pluripotent stem cells with the base medium comprising CHIR99021 so that the vertebrate pluripotent stem cells differentiate into presomitic mesoderm cells;
on about day 4, removing the base medium comprising CHIR99021 and culturing the presomitic mesoderm cells in base medium comprising fibroblast growth factor 9 (FGF9) and heparin so that the presomitic mesoderm cells differentiate into intermediate mesoderm cells;
on about day 7, removing the medium comprising FGF9 and heparin and culturing the cells.

3. The method of claim 1, wherein the step of treating the genital ridge cells with a culture medium comprising fibroblast growth factor 9 (FGF9), insulin and/or IGF1 so that the genital ridge cells differentiate into Sertoli cells further comprises on about days 7 to 10, removing the base medium and culturing the genital ridge cells in base medium comprising insulin-like growth factor 1 (IGF1), insulin and FGF9 so that the genital ridge cells differentiate into artificial Sertoli cells.

4. The method claim 1, wherein the step of treating the genital ridge with a base medium comprising fibroblast growth factor 9 (FGF9), insulin and/or IGF1 so that the genital ridge cells differentiate into Sertoli cells further comprises treating the genital ridge cells with epidermal growth factor (EGF), bone morphogenetic protein 4 (BMP4), IWR1 or combinations thereof.

5. The method of claim 1, wherein the step of treating the genital ridge with a base medium comprising fibroblast growth factor 9 (FGF9), insulin and/or IGF1 so that the genital ridge cells differentiate into Sertoli cells further comprises treating the genital ridge cells with follicle stimulating hormone (FSH) and/or luteinizing hormone and/or testosterone or a combination thereof.

6. The method of claim 1, wherein the vertebrate pluripotent stem cells are human embryonic stem cells (hESC).

7. An in vitro method for production of Sertoli cells from vertebrate pluripotent stem cells comprising:
deriving anterior intermediate mesoderm cells from pluripotent stem cells;
treating the anterior intermediate mesoderm cells with a base medium comprising insulin and/or IGF1 so that the anterior intermediate mesoderm cells differentiate into Sertoli cells.

8. The method of claim 7, wherein the step of deriving anterior intermediate mesoderm cells further comprises:
providing vertebrate pluripotent stem cells in a maintenance medium comprising a ROCK inhibitor;
at day 0, removing the maintenance medium comprising a ROCK inhibitor and culturing the vertebrate pluripotent stem cells with the base medium comprising CHIR99021 so that the vertebrate pluripotent stem cells differentiate into presomitic mesoderm cells; and
on about day 4, removing the base medium comprising CHIR99021 and culturing the presomitic mesoderm cells in base medium comprising fibroblast growth factor 9 (FGF9) and heparin so that the presomitic mesoderm cells differentiate into intermediate mesoderm cells.

9. The method of claim 7, wherein the step of treating the intermediate mesoderm cells with a culture medium comprising insulin and/or IGF1 so that the intermediate mesoderm cells differentiate into Sertoli cells further comprises on about day 7, removing the medium comprising FGF9 and heparin and culturing the intermediate mesoderm cells in base medium comprising insulin-like growth factor 1 (IGF1) and insulin so that the intermediate mesoderm cells differentiate into artificial Sertoli cells.

10. The method of claim 7, wherein the step of treating the anterior intermediate mesoderm cells with a base medium comprising insulin and/or IGF1 further comprises treating the genital ridge cells with FGF9, epidermal growth factor (EGF), bone morphogenetic protein 4 (BMP4), IWR1, or combinations thereof.

11. The method of claim 7, wherein the step of treating the anterior intermediate mesoderm cells with a base medium comprising insulin and/or IGF1 further comprises treating the genital ridge cells with follicle stimulating hormone (FSH) and/or luteinizing hormone and/or testosterone or combinations thereof.

12. The method of claim 7, wherein the vertebrate pluripotent stem cells are human embryonic stem cells (hESC).

13. The method of claim 7, wherein the wherein the step of deriving anterior intermediate mesoderm cells further comprises:
providing vertebrate pluripotent stem cells in a maintenance medium comprising LIF;
at day 0, removing the maintenance medium comprising LIF and culturing the vertebrate pluripotent stem cells with the base medium comprising Activin A and bFGF so that the vertebrate pluripotent stem cells differentiate into epiblast cells; and
on about day 2, removing the base medium comprising Activin A and bFGF and culturing the epiblast cells in base medium comprising Activin A and RA so that the epiblast cells differentiate into intermediate mesoderm cells.

14. The method of claim 7, wherein the step of treating the intermediate mesoderm cells with a culture medium comprising insulin and/or IGF1 so that the intermediate mesoderm cells differentiate into Sertoli cells further comprises on about day 4, removing the medium comprising Activin A and RA and culturing the intermediate mesoderm cells in base medium comprising insulin-like growth factor 1 (IGF1) and insulin so that the intermediate mesoderm cells differentiate into artificial Sertoli cells.

15. The method of claim 7, wherein the vertebrate pluripotent stem cells are mouse embryonic stem cells (mESC).

16. The method of claim 7, wherein the artificial Sertoli cells express at least one of the markers selected from the group consisting of EMX2, WT, SOX9, and LHX9.

17-22. (canceled)

23. The method of claim 7, wherein the base medium comprising insulin-like growth factor 1 (IGF1) and insulin further comprises one or more of retinoic acid, PDG₂, and FGF9, and preferably a combination thereof.

24. (canceled)

25. The method of claim 7, further comprising culturing the artificial Sertoli cells under conditions such that the artificial Sertoli cells form an artificial Sertoli cell organoid.

26. (canceled)

27. (canceled)

28. The method of claim 7, further comprising the step of isolating the artificial Sertoli cells.

29. The method of claim 28, further comprising transplanting the isolated artificial Sertoli cells into a mammal.

30-59. (canceled)
